(19) United States
(12) Patent Application Publication
(10) Pub. No.: US 2018/0000817 A1
(43) Pub. Date: Jan. 4, 2018

(54) TREATMENT OF HUTCHINSON-GILFORD PROGERIA SYNDROME AND DISEASES RELATED TO VASCULAR AGEING

(71) Applicant: BIOCANT - ASSOCIAÇÃO DE TRANSFERÊNCIA DE TECNOLOGIA, Cantanhede (PT)

(72) Inventors: Lino DA SILVA FERREIRA, Coimbra (PT); Patricia Raquel PINHEIRO PITREZ PEREIRA, Braga (PT); Helena Sofia ESMERALDO DE CAMPOS VAZÃO, Alcobaça (PT)

(21) Appl. No.: 15/543,703

(22) PCT Filed: Jan. 15, 2016
(86) PCT No.: PCT/IB2016/050208

§ 371 Date: Jul. 14, 2017

(30) Foreign Application Priority Data
Jan. 15, 2015 (PT) 108150

(51) Int. Cl. A61K 31/505; A61K 31/16; A61K 31/381; A61K 9/00; A61K 31/54; C12N 15/113; G01N 33/68; G01N 33/50; C12Q 1/68; A61K 31/506

(52) U.S. Cl. CPC A61K 31/505; C12Q 1/6883; A61K 31/16; A61K 31/381; A61K 31/506; A61K 31/54; C12N 15/1137; G01N 33/6893; G01N 33/5061; C12Y 304/24; A61K 9/0019; C12Q 2600/178; C12N 2320/35; C12N 2310/14; C12Q 2600/136; G01N 2333/96486; G01N 2800/385

(57) ABSTRACT

The present disclosure relates to the treatment of Hutchinson-Gilford Progeria Syndrome (HGPS) and diseases related to vascular ageing and in the treatment of smooth muscle cells diseases, in particular an inhibitor of a metalloprotease the treatment of smooth muscle cells diseases. The disclosure subject matter describes a more effective therapies for the treatment of Hutchinson-Gilford Progeria Syndrome and diseases related to vascular ageing, or namely by the use of an inhibitor of a metalloprotease.

### TECHNICAL FIELD

The present disclosure relates to the treatment of Hutchinson-Gilford Progeria Syndrome (HGPS) and diseases related to vascular ageing and in the treatment of smooth muscle cells diseases, in particular an inhibitor of a metalloprotease.

The disclosure subject matter describes a more effective therapies for the treatment of Hutchinson-Gilford Progeria Syndrome and diseases related to vascular ageing namely by the use of an inhibitor of a metalloprotease.

### TECHNICAL BACKGROUND

HGPS is a rare, progressive ageing disease in children that leads to premature death. Smooth muscle cells (SMCs) are the most affected cells in HGPS patients, although the reason for such sensitivity remains poorly understood.

HGPS is caused by a single mutation of the lamin A gene (LMNA) resulting in the generation of an abnormal lamin A named “progerin”. Progerin lacks the proteolytic cleavage site normally used to remove the farnesylated carboxy terminus from lamin A during posttranslational processing. Progerin accumulates with successive cell passage number, leading to progressive nuclear envelope deformations and invaginations, and premature senescence. In general, patients die because of myocardial infarction or stroke. In the last years, pre-clinical (Cao K, Graziotto J J, Blair C D, Mazzulli J R, Erdos M R, Krainc D, Collins F S. Rapamycin reverses cellular phenotypes and enhances mutant protein clearance in hutchinson-gilford progeria syndrome cells. Science translational medicine. 2011;3:89ra58) and clinical treatments (Gordon L B, Kleinman M E, Miller D T, Neuberg D S, Giobbie-Hurder A, Gerhard-Herman M, Smoot L B, Gordon C M, Cleveland R, Snyder B D, Fligor B, Bishop W R, Statkevich P, Regen A, Sonis A, Riley S, Ploski C, Correia A, Quinn N, Ullrich N J, Nazarian A, Liang M G, Huh S Y, Schwartzman A, Kieran M W. Clinical trial of a farnesyltransferase inhibitor in children with hutchinson-gilford progeria syndrome. Proc Natl Acad Sci USA. 2012;109:16666-16671) have been proposed for HGPS patients; however, further efforts are needed in the design of more effective therapies.

One of the hallmarks of the disease is the loss of SMCs in the medial layer of large arteries with replacement by collagen and extracellular matrix, and in many cases calcification. Studies in transgenic mice carrying the G608G mutated human lamin A showed progressive loss of SMCs, elastic fiber breakage, thickening of the adventitia and medial layer, accumulation of hyaluronan and collagen. Although lamin A and progerin were expressed in several tissues in the transgenic mice, progeria phenotype is essentially observed in SMCs. This agrees with the fact that children with HGPS die because of progressive arterial occlusion. The mechanism underlying the vulnerability of the SMCs to arterial flow (mechanical stress) remains poorly understood, in part due to the difficulty of having SMCs from HGPS patients.

Induced pluripotent stem cells (iPSCs) offer an unlimited source of SMCs to study the HGPS disease, including from a developmental point of view. Three recent studies have generated iPSCs from fibroblasts obtained from patients with HGPS. Strikingly, HGPS-iPSCs have low expression of lamin A/C and progerin proteins in a pluripotent state; however, the expression of progerin could be reactivated after the differentiation of HGPS iPSCs into different types of cells including fibroblasts, mesenchymal stem cells, endothelial cells, SMCs but not in neural progenitors. The differentiated cells showed nuclear dysmorphology, cell growth retardation, susceptibility to apoptosis, proliferation reduction, DNA-repair defects and reduced telomere length. Two studies have reported the differentiation of HGPS iPSCs into SMCs. One of the studies has derived SMCs from iPSC-derived mesenchymal stem cells (MSCs). In the other study, Progeria iPSCs were differentiated into SMCs in a co-culture with OP9 cultures for 10 days Unfortunately, in both studies, it is unclear the differences in the differentiation profile of the HGPS iPSCs as compared to unaffected iPSCs (N-iPSCs), in terms of SMC-marker expression, SMC maturation, functionality and response to shear stress.

These facts are disclosed in order to illustrate the technical problem addressed by the present disclosure.

### GENERAL DESCRIPTION

In view of the drawbacks to the prior art, the disclosure subject matter describes a more effective therapies for the treatment of Hutchinson-Gilford Progeria Syndrome (HGPS) or diseases related to vascular ageing, or for use in the treatment or diagnostic of smooth muscle cells diseases, in particular an inhibitor of a metalloprotease.

The disclosure subject matter is related to an inhibitor of a metalloprotease for use in the treatment of Hutchinson-Gilford Progeria Syndrome or in the treatment of vascular ageing diseases, in particular the metalloprotease is a zinc endopeptidases.

In an embodiment the inhibitor could be an inhibitor of MMP-1, MMP-2, MMP-3, MMP-7, MMP-9, MMP-13, or MMP-14, among others; preferably MMP-1, MMP-7, MMP-13 or MMP-14; more preferably MMP-13.

In other embodiment the inhibitor could be select from the compounds of the following list:
pyrimidine-4,6-dicarboxylic acid, bis-(4-fluoro-3-methyl-benzylamide)—MMP 13 inhibitor;
pyrimidine-4,6-dicarboxylic acid, bis-(3-methyl-benzylamide);
pyrimidine-4,6-dicarboxylic acid, bis-(benzylamide);
pyrimidine-4,6-Dicarboxylic Acid Bis-[(Pyridin-3-YL-Methyl)-Amide;
(2R,3S)-N4-Hydroxy-2-isobutyl-N1-[(2S)-1-(methylamino)-1-oxo-3-phenyl-2-propanyl]-3-[(2-thienylsulfanyl)methyl]succinamide—Batimastat BB 94;
N-[2,2-dimethyl-1-(methylcarbamoyl)propyl]-2-[hydroxy-(hydroxycarbamoyl)methyl]-4-methyl-pentanamide—Marimastat;
N-hydroxy-4-((4-((4-hydroxy-2-butynyl)oxy)phenyl)sulfonyl)-2,2-dimethyl-3-thiomorpholinecarboxamide—Apratastat;
MMP-13 siRNA from Santa Cruz Biotechnology; or mixtures thereof; among others.

Other aspect of the present invention relates to inhibitor of a metalloprotease for use in the treatment of smooth muscle cells diseases wherein said inhibitor is select from the compounds of the following list:
pyrimidine-4,6-dicarboxylic acid, bis-(4-fluoro-3-methyl-benzylamide);
pyrimidine-4,6-dicarboxylic acid, bis-(3-methyl-benzylamide);
pyrimidine-4,6-dicarboxylic acid, bis-(benzylamide);
pyrimidine-4,6-Dicarboxylic Acid Bis-[(Pyridin-3-YL-Methyl)-Amide;
(2R,3S)-N4-Hydroxy-2-isobutyl-N1-[(2S)-1-(methylamino)-1-oxo-3-phenyl-2-propanyl]-3-[(2-thienylsulfanypmethyl]succinamide;
N-[2,2-dimethyl-1-(methylcarbamoyl)propyl]-2-[hydroxy-(hydroxycarbamoyl)methyl]-4-methyl-pentanamide;
N-hydroxy-4-((4-((4-hydroxy-2-butynyl)oxy)phenyl)sulfonyl)-2,2-dimethyl-3-thiomorpholinecarboxamide;
MMP-13 siRNA; or mixtures thereof; among others.

The disclose subject matter also relates to a pharmaceutical composition comprising at least one metalloprotease inhibitor as described in any one of the previous claims in a therapeutically effective amount and a pharmaceutically acceptable carrier, adjuvant, excipient, carrier or mixtures thereof.

Another embodiment the pharmaceutical composition could be an injectable formulation, in particular an intraperitoneal injection.

In another embodiment the pharmaceutical composition the inhibitor concentration may vary between 5 nM-7000 nM, preferably 5 nM-240 nM, more preferably 5 nM-100 nM; more preferably 5 nM-50 nM, more preferably 8 nM-20 nM.

In an embodiment, the daily form consists of ampoule or injection or other device, comprising a definitive amount of metalloprotease inhibitor, the whole of which is intended to be administered as a single dose.

The disclose subject matter also relates to a kit for use in drug screening for the treatment/diagnostic of Hutchinson-Gilford Progeria Syndrome or for use in the treatment/diagnostic of vascular ageing diseases or for use in the treatment or diagnostic of smooth muscle cells diseases, comprising:
at least a metalloprotease inhibitor described in any of the previous claims;
a fluidic system suitable use in screening therapeutic drugs;
a fluidic system suitable for use in screening therapeutic drugs;
a Hutchinson-Gilford Progeria Syndrome smooth muscle cells population.

Throughout the description and claims the word “comprise” and variations of the word, are not intended to exclude other technical features, additives, components, or steps. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### DEFINITIONS

“CD34+ cells” refers to cells expressing CD34 antigen. This antigen is a single-chain transmembrane glycoprotein expressed in several cells including human hematopoietic stem and progenitor cells, vascular endothelial cells, embryonic fibroblasts and some cells in fetal and adult nervous tissue.

A metalloproteinase, or metalloprotease, is any protease enzyme whose catalytic mechanism involves a metal.

A matrix metalloproteinases (MMPs) are a family of zinc endopeptidases that degrade proteins of the extracellular matrix, including collagens, elastins, matrix glycoproteins, and proteoclycans.

### DETAIL DESCRIPTION

The present disclosure concerns the reasons of HGPS-SMCs vulnerability using induced pluripotent stem cells (iPSCs) obtained from HGPS fibroblast patients. SMCs differentiated from HGPS-iPSCs showed impaired maturation as confirmed by a low expression of calponin and SMMHC genes and individualized calponin fibers. HGPS-iPSC SMCs shared similar features observed on progerin-expressing cells such as activation of several effectors of NOTCH signaling pathway and response to farnesyltransferase inhibitors. When HGPS-iPSC SMCs are cultured under arterial flow conditions they show an up-regulation of progeria and osteogenic markers followed by their detachment from the culture substrate. Yet, HGPS-iPSC SMC detachment is prevented by the inhibition of MMP-13. This finding opens new opportunities for the treatment of HGPS disease and diseases related to vascular ageing.

In an embodiment, it is disclosed that SMCs derived from HGPS-iPSCs have lower levels of maturation than SMCs derived from N-iPSCs and they showed activation of several effectors of the NOTCH signaling pathway, which induced an osteogenic differentiation program. These cells detach from the substrate in arterial flow conditions and the kinetics of this process is dependent on the percentage of progerin-expressing cells. It is further shown that the chemical inhibition of MMP13 decreased significantly SMC detachment. The solution disclosed open new possibilities for new therapies in particular the treatment of HGPS patients.

### BRIEF DESCRIPTION OF THE DRAWINGS

The following figures provide preferred embodiments for the present disclosure and should not be seen as limiting the scope of the disclosure.

FIG. 1—Derivation and characterization of SMCs from N-iPSCs and HGPS-iPSCs;
(A) Expression of progeria (progerin and lamins) and SMC (α-SMA, SMα-22) markers on undifferentiated HGPS-iPSCs and N-iPSCs, as evaluated by qRT-PCR analyses. HGPS fibroblasts and hVSMCs were used as controls. Results are Mean±SEM (n=4). *,**,***,**** denotes statistical significance (P<0.05, P<0.01, P<0.001, P<0.0001);
(B) Schematic representation of the methodology used to differentiate iPSCs into SMCs;
(C) Expression of SMC markers on N-iPSC SMCs and HGPS-iPSC SMCs; (C.1) Gene expression by qRT-PCR. The expression of each gene was normalized by the expression of GAPDH and then by the gene expression observed in hVSMCs. Percentage of positive cells expressing SMC markers (C.2) and organized fibers (C.3) as evaluated by imunofluorescence (at least 100 cells were counted per each marker). Results are Mean±SEM (n=3). **,*** denotes statistical significance (P<0.01, P<0.001);
(D) Expression of progeria markers on N-iPSC SMCs and HGPS-iPSC SMCs. (D.1) Gene expression by qRT-PCR (gene expression was normalized by the housekeeping gene GAPDH). HGPS fibroblasts were used as control. (D.2) Percentage of cells positive for progerin as assessed by imunofluorescence. Results are Mean±SEM (n=3). *,**,*** denotes statistical significance (P<0.05, P<0.01, P<0.001);
(E) Immunofluorescence analysis performed on HGPS-iPSC SMCs (clone 1) and N-iPSC SMCs at passage 8 for SMC and progerin markers. Nuclei were visualized with DAPI stain (blue). Scale bar is 20 μm;
(F) Intracellular Ca²⁺ measurements on N-iPSC SMCs and HGPS-iPSC SMCs loaded with FURA-2 and exposed to two agonists (100 μM histamine and 10⁻⁵ M angiotensin). hVSMCs and HGPS fibroblasts were used as controls;
(G) Contractility of N-iPSC SMCs and HGPS-iPSC SMCs after exposure to carbachol (10⁻⁵ M in DMEM medium, for 30 min) or atropine (10⁻⁴ M, 1 h) plus carbachol (10⁻⁵ M for 30 min). hVSMCs and HACAT were used as controls. Results are Mean±SEM (n=8). *** denotes statistical significance (P<0.001).

FIG. 2—Activation of signaling pathways during the differentiation of HGPS-iPSCs into SMCs;
(A) Expression of NOTCH upstream and downstream effector genes;
(B) Expression of osteogenic genes;
(C) Expression of WNT signaling pathway genes;
   evaluated by qRT-PCR during differentiation of CD34+ cells into SMCs. The differentiation methodology is described in FIG. 1B. In A-C, values were normalized to the housekeeping gene GADPH. Results are Mean±SEM (n=4). *,***,**** denotes statistical significance (P<0.05, P<0.01, P<0.001, P<0.0001, respectively).

FIG. 3—Vulnerability of HGPS-iPSC SMCs to arterial flow conditions. Cells were cultured for 6-8 days in arterial flow conditions (20 dyne/cm²);
(A) Light microscopy images of HGPS-fibroblasts, hVSMCs or HGPS-iPSC SMCs (clone 1) at different culture days. Only HGPS-iPSC SMCs detached from the microfluidic system at day 4;
(B) Number and area of cell clumps in HGPS-iPSC SMCs (clone 1) at different times (at least 3 images (×10) have been quantified per time);
(C) Number of cells per surface area (mm²) during cell culture under arterial flow (at least 3 images (×10) have been quantified per time);
(D) Cell metabolism evaluated by Presto Blue;
(E) Expression of nuclear proliferation marker, Ki67 (at least 3 images (×10) have been quantified per time). The percentage of Ki67 positive cells were analyzed by imunofluorescence
(F) Apoptosis assessed by a luminescence assay that evaluates caspase-9 activity. Results were normalized by cell number;
(G) Gene expression of HGPS markers (lamin A, lamin B and progerin), as evaluated by qRT-PCR, in N-iPSC SMCs and HGPS-iPSC SMCs. Gene expression was normalized by the housekeeping gene GAPDH. Gene expression in HGPS fibroblasts is represented as a dashed line;
(H) Cellular expression of progeria markers (blebbing, dysmorphic nuclei, progerin and lamin A) as evaluated by imunofluorescence (at least 100 cells were counted). The expression of progeria markers in HGPS fibroblasts is represented in the graph by a dashed line;
(I) Cellular expression of SMC markers (α-SMA, SMMHC, Calp and SMα-22) as evaluated by qRT-PCR. Gene expression was normalized by the housekeeping gene GAPDH. (J) Percentage of cells with organized calponin fibers;
   From B to J, results are Mean±SEM [B-F: n=3; H: n=3; G: n=4; I: n=4; J: n=5]. *,**,***,**** denotes statistical significance (P<0.05, P<0.01, P<0.001, P<0.0001).

FIG. 4—Characterization of the osteogenic differentiation program;
(A) Expression of osteogenic markers (RUNX2 and BMP2) in HGPS-iPSC SMCs and N-iPSC SMCs cultured under flow conditions, as evaluated by qRT-PCR analysis. Gene expression was normalized by the housekeeping gene GAPDH;
(B) Expression of alkaline phosphatase normalized by cell number per mm². hVSMC and HGPS-fibroblasts were used as controls;
(C) Expression of osteopontin (OPN). The intensity of OPN was measured and data normalized by cell number;
(D) Expression of Alkaline phosphatase, normalized by cell number per mm², in cells cultured under flow conditions in osteogenic media;
(E) Mineralization on cells cultured under flow conditions in osteogenic media as assessed by Alizarin red staining. (i) Primary osteoblasts, (ii) hVSMCs, (iii) N-iPSC SMCs and (iv) HGPS-iPSC SMCs (clone 1). Scale bar 200 μm;
(F) Effect of calcification inhibitor (PPi) in HGPS-iPSC SMC (clones 1 and 2) detachment. The number of cells was evaluated after 7 and 12 days under arterial flow and was normalized by the surface area (mm²); In graphs A, B, C, D, and F results are Mean±SEM, n=3. *,**,***,**** denotes statistical significance (P<0.05, P<0.01, P<0.001, P<0.0001).

FIG. 5—Genes expression profile on HGPS-iPSC SMCs cultured under flow;
(A) Microarray analysis of mRNA profile in HGPS-iPSC-SMC (clone 1) using N-iPSC-SMC as control. Identification of molecular hits through gene ontology. Right panel: biological processes. Left panel: signaling pathways;
(B) qRT-PCR validation for 16 genes with fold-changes greater than 3. Fold change was between day 0 and day 4;
(C) Quantification of general MMP activity (intracellular) by a fluorescence kit. HGPS-iPSC SMCs, hVSMCs and N-iPSC SMCs were analyzed at day 0 and day 4 (in this case also in the presence of a MMP13 inhibitor) under flow. The fluorescence signal was normalized on cell number for each condition;
(D) Expression of WNT3a and WNT7a genes for HGPS-iPSC-SMC and N-iPSC-SMC cultured under flow conditions, as evaluated by qRT-PCR analyses. Gene expression was normalized by the housekeeping gene GAPDH;
(E) Effect of MMP13 or BB94 inhibition in HGPS-iPSC SMC detachment. The number of cells was evaluated after 7 and 12 days under arterial flow and was normalized by the surface area (mm²);
(F) Effect of HGPS-iPSC SMC (clone 1) (F.1) or N-iPSC SMCs conditioned media (F.2) (in both cases obtained after 4 days under flow conditions) on hVSMCs cultured under flow conditions;
(G) Percentage of progerin positive cells and alkaline phosphatase normalized by cell number per mm², after 7 days under flow conditions with SmGM2 media supplemented with MMP-13 inhibitor;
   In graphs B, C, D, E and F, results are Mean±SEM. In (B) and (D) n=4, in (C) n=3 and in (E) n=9. *,**,***,**** denotes statistical significance (P<0.05, P<0.01, P<0.001, P<0.0001).

FIG. 6—Inhibition of MMP-13 by siRNA; (A) HGPS-iPSC SMCs (clone 2) were transfected with siRNA MMP-13. The relative expression of MMP-13 was assessed by qRT-PCR (A.1). These cells were cultured under flow conditions and cell detachment evaluated (A.2). Results are Mean±SEM. *,**,***,**** denotes statistical significance (P<0.05, P<0.01, P<0.001, P<0.0001).

FIG. 7—Vulnerability of SMCs obtained from progeria mice to arterial flow conditions. mSMC from wild-type, heterozygous Lmna^{G609G/+} and homozygous Lmna^{G609G/G609G} mice were isolated at 6 or 18 weeks-old. (A.1-A.2) Expression of SMC (A.1) and HGPS (A.2) markers as calculated by immunofluorescence. Cells were cultured in static conditions. (A.3) Expression of HGPS markers in cells cultured under shear stress, as evaluated by immunofluorescence. (A.4) % of cell detachment under arterial flow (at least 3 images (×10) have been quantified per time). Results are Mean±SEM. *,**,***,**** denotes statistical significance (P<0.05, P<0.01, P<0.001, P<0.0001).

FIG. 8—Expression of progeria and SMC markers in CD34+ cells; (A) Characterization of CD34+, at passage 1. Gene expression of lamins (lamin A, lamin B1) and progerin (A.1). Gene expression of SMC markers (A.2). In A.1 and A.2, results are Mean±SEM (n=4). *,**,***,**** denotes statistical significance (P<0.05, P<0.01, P<0.001, P<0.0001). N-iPSCs refers to iPSCs without the disease state; HGPS-iPSCs refers to iPSCs derived from skin fibroblasts of one HGPS patient (clone 1); and hVSMCs refers to normal human vascular smooth muscle cells. Calp is the abbreviation of calponin.

FIG. 9—Expression of progeria and SMC markers after 4 passages using SMC inductive media. (A) Expression of lamins (lamin A, lamin B1; A.1), progerin (A.1) and SMC (A.2) gene markers in cells differentiated from N- or HGPS-iPSCs (clone 1) for 4 passages (ca. 15 days) in inductive media. Gene expression was normalized by the expression of housekeeping gene GAPDH. Results are Mean±SEM (n=4). * denotes statistical significance (P<0.05). (B) Percentage of cells that express SMC proteins at the inductive stage of cell differentiation. Results are Mean±SEM (n=4). (C) Percentage of cells with organized α-SMA and calponin fibers (at least 100 cells were counted). Results are Mean±SEM (n=4).

FIG. 10—Expression of progeria and SMC markers in HGPS-iPSCSMCs (clone 2). (A) Progeria markers characterization. (A.1) Expression of progeria proteins by immunofluorescence. (A.2) Expression of progeria gene markers. (A.3) Expression of progeria markers (proteins and morphology). In (A.2) and (A.3), results are Mean±SEM (n=4). *,**,***,**** denotes statistical significance (P<0.05, P<0.01, P<0.001, P<0.0001);
(B) SMC markers characterization. (B.1) Expression of SMC proteins by immunofluorescence. (B.2) Expression of SMC gene markers. (B.3) Percentage of cells that express SMC markers at protein level. (B.4) Percentage of cells with organized α-SMA and calponin fibers. In (B.2), results are Mean±SEM (n=4). In (B.3) and (B.4), results are Mean±SEM (n=5);
(C) Osteogenic markers characterization. Osteopontin protein was evaluated by imunofluorence. Differences between HGPS-iPSC-SMC (clone 2) and N-iPSC-SMC. Calcium deposits were also assessed by alizarin red staining. Scale bar=−−−−μm;
(D) Number of cells per surface (mm²) during cell culture under flow (at least 3 images (×10) have been quantified per time). Time under flow shear stress in minutes.

FIG. 11—Expression of NOTCH, WNT and osteogenic markers on HGPS-iPSC-SMCs (clone 2);
(A) Expression of NOTCH signaling evaluated by qRT_PCR, during SMC differentiation. Comparison between HGPS-iPSC SMCs (clone 2) and N-iPSCs;
(B) Expression of osteogenic markers (RUNX2 and BMP2) on HGPS-iPSC SMCs (clone 2), as assessed by qRT-PCR;
(C) WNT signaling markers (WNT3a and WNT7a) evaluated by qRT-PCR. Gene expression was normalized by the housekeeping gene GADPH.
   Results are Mean±SEM (n=4). *,**,***,**** denotes statistical significance (P<0.05, P<0.01, P<0.001, P<0.0001).

FIG. 12—Response of HGPS-iPSC SMCs to a farnesyltransferase inhibitor, tipifarnib;
(A) Accumulation of prelamin A in HGPS-iPSC SMCs treated with tipifarnib;
(B) Nuclear abnormalities (B) in HGPS-iPSC SMCs cultured with tipifarnib;
(C) Nuclear blebbing in HGPS-iPSC SMCs cultured with tipifarnib;
(D) Expression of alkaline phosphatase in HGPS-iPSC SMCs, N-IPSC-SMCs and hVSMCs cultured in static conditions;
(E) Effect of tipifarnib in the expression of alkaline phosphatase;
(F) Effect of lonafarnib and tipifarnib in cell number cultured under flow conditions.
   In A-F results are Mean±SEM (n=4 or n=2). *,**,*** denotes statistical significance (P<0.05, P<0.01, P<0.001).

FIG. 13—Effect of progerin inhibition on SMC vulnerability;
(A) HGPS-iPSC SMCs (clone 2) were transfected with control siRNA or siRNA to knock-down the expression of progerin. (A.1) The expression of progerin gene before and after transfection was performed by qRT-PCR. Gene expression was normalized by the housekeeping gene GAPDH. Results are Mean±SEM (n=4). ** denotes statistical significance (P<0.01). (A.2) The percentage of cells positive for progerin staining was evaluated before and after transfection. Results are Mean±SEM (n=7-8). *** denotes statistical significance (P<0.001). (A.3) Expression of MMPs per each SMC after transfection. Results are Mean±SEM (n=6). (A.4) Cell viability evaluated by Presto Blue, in cells cultured under flow, at different times after siRNA transfection. Results are Mean±SEM (n=2-3).

FIG. 14—Expression of NOTCH and Wnt signaling pathways on HGPS-iPSC-SMCs (clone 1) cultured under flow conditions;
(A) Expression of NOTCH signaling effectors as evaluated by qRT_PCR, in HGPS-iPSC-SMC (clone 1) and N-IPSC-SMCs cultured under flow conditions;
(B) Expression of Wnt signaling markers (Wnt 3a and Wnt 7a) as evaluated by qRT-PCR, in HGPS-iPSC-SMCs (clone 1) and N-iPSC-SMCs cultured under flow conditions.
   Results are Mean±SEM (n=4). *,**,*** denotes statistical significance (P<0.05, P<0.01, P<0.001, P<0.0001).

FIG. 15—Expression of H2AX, a marker for DNA damage. For quantification, 3 slides have used per condition. In each slide, 3-4 images were analysed and more than 100 cells counted for the presence of H2AX foci. Results are Mean±SEM (n=4).

FIG. 16—Effect of ECM on SMC vulnerability. (A.1) Decellularized ECM from hVSMC was used to seed HGPS-iPSC SMCs (clone 1). After 19 hours HGPS-iPSC-SMC completely detached from the ECM. (A.2) HGPS-iPSC SMCs (clone 1 and 2) were seeded on mitotically-inhibited hVSMCs and exposed to flow conditions. Cell viability was evaluated by Presto Blue. After 2 days, most of the cells are already detached. Results are Mean±SEM (n=2-3).

FIG. 17—Vulnerability of mouse SMCs to arterial flow conditions. Mouse SMCs were cultured for 9-26 days in arterial flow conditions (120 dyne/cm2). (A) Imunofluorescence analysis performed on mouse SMCs (6-week-old wild-type and homozygous Lmna G609G/G609G mice) at passage 4 for α-SMA and Lamin A. Nuclei was stained with DAPI. Scale bar is 20 μm. (B) Percentage of dysmorphic nuclei, nuclei blebbing and SMC organized fibers in mSMCs (assessed in static conditions). (C) Percentage of adhered cells over time. Cells were cultured under shear stress conditions. Results are Mean±SEM [B: n=4; C: n=3]. **** denotes statistical significance (P<0.0001).

FIG. 18—Effect of progerin inhibition on SMC vulnerability. HGPS-iPSC SMCs (clone 1) were seeded overnight in the microfluidic system and then perfused with SmGM-2 medium supplemented with PMOs (Ex10 and Ex11 at 20 μM each) at arterial flow rate (20 dyne/cm2). After 48 h the medium was replaced by new medium supplemented with PMOs. Non-treated cells were used as negative control. (A) Cell morphology and number observed by light microscopy. (A.1) Non-treated cells 4 days under flow conditions; (A.2) HGPS-iPSC SMCs treated with PMOs 4 days (A.2) and 8 days (A.3) under flow conditions. (B) Number of cells per surface area (mm2) (at least 6 images (×20) have been quantified per condition). Results are Mean±SEM (n=4). (C) Progerin gene expression 4 days under flow conditions evaluated by qRT-PCR. Gene expression was normalized by the housekeeping gene GAPDH. Results are Mean±SEM (n=4). *** denotes statistical significance (P<0.001).

FIG. 19—MMP13 activity in HGPS-iPSC SMCs cultured under flow shear stress. (B.1) Quantification of general MMP activity (intracellular). Cells were analyzed at day 0 and day 4 under flow. Fluorescence signal was normalized by cell number. (B.2) Quantification of MMP13 activity (cell culture media) by ELISA. Cells were analyzed at day 0 and day 4 under flow. Fluorescence signal was normalized by cell number. (C) Quantification of MMP13 in HOZ mSMCs and WT mSMCs. Cells were analyzed at day 0 and day 8 under flow. Fluorescence signal was normalized by cell number. Results are Mean±SEM. In (C) n=3 and in (D) n=9. *,***,**** denotes statistical significance (P<0.05, P<0.001, P<0.0001).

FIG. 20—MMP13 inhibition in HGPS-iPSC SMCs cultured under flow shear stress; (C) Percentage of progerin positive cells after 7 days under flow conditions with SmGM2 media supplemented or not with MMP13 inhibitor. (D) Percentage of adherent HOZ mSMCs after culture in flow conditions with media supplemented or not with MMP13 or BB94 inhibitors. Results are Mean±SEM. ***,**** denotes statistical significance (P<0.001, P<0.0001).

FIG. 21—MMP inhibition by BB94 significantly increases SMC number in aortic arch of LmnaG609G/G609G mice. (A) Heart rate of LmnaG609G/G609G mice treated or not with BB94. (B) Imunofluorescence analysis performed on mouse SMC for α-SMA showing higher number of mSMCs in treated aortic arch. Cell nuclei were stained with DAPI. SMCs were stained for α-SMA. Scale bar is 20 μm. (C) Number of SMC nuclei in aortic arch per tissue area (mm2) in LmnaG609G/G609G mice treated or not with BB94. (D) Expression of SMC genes in aortic arches of LmnaG609G/G609G mice treated or not with BB94. Gene expression was normalized by the housekeeping gene GAPDH. Results are Mean±SEM. ***** denotes statistical significance (P<0.01, P<0.001).

SMCs derived from HGPS-iPSCs express progerin and are functional—Skin-derived fibroblast cultures from two HGPS patients were obtained from Coriell Institute. iPSCs were generated and characterized as previously described (from now on termed HGPS-iPSCs (clone 1 and clone 2)). iPSCs without the disease state (N-iPSCs) were obtained from fibroblasts or cord blood. HGPS fibroblasts from Coriell and healthy human vascular smooth muscle cells (hVSMCs) from Lonza were used as controls. Initially, the expression of genes related to lamins (A and B1) and progerin in these undifferentiated cells was characterized. Previous studies have shown that lamin A is expressed on differentiated SMCs but low expressed in undifferentiated iPSCs. In contrast, lamin B1 is highly expressed in undifferentiated iPSCs but low expressed in differentiated SMCs. In agreement with previous data, undifferentiated iPSCs express low levels of progerin and lamin A mRNA, and high levels of lamin B1, as assessed by quantitative RT-PCR analyses (FIG. 1A).

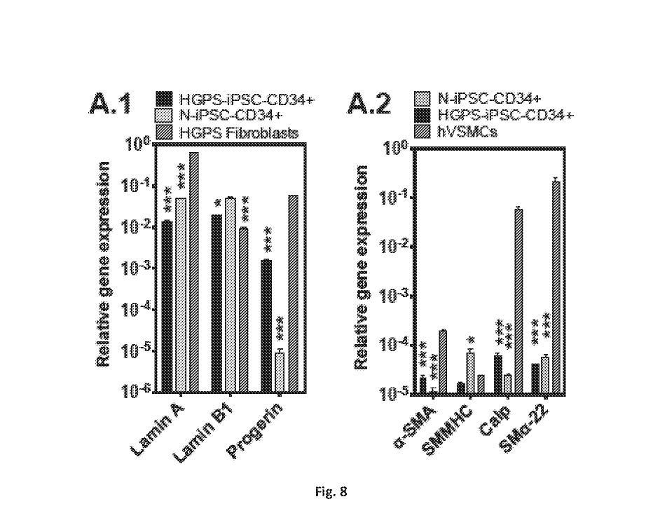

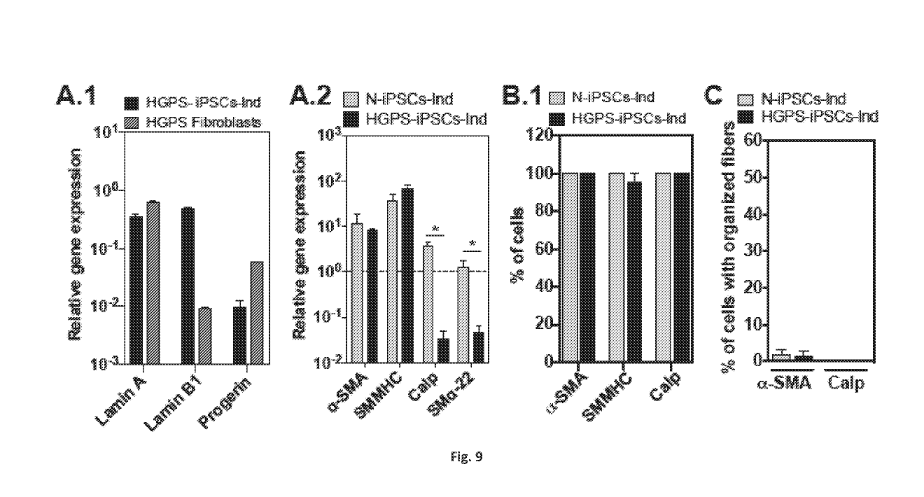

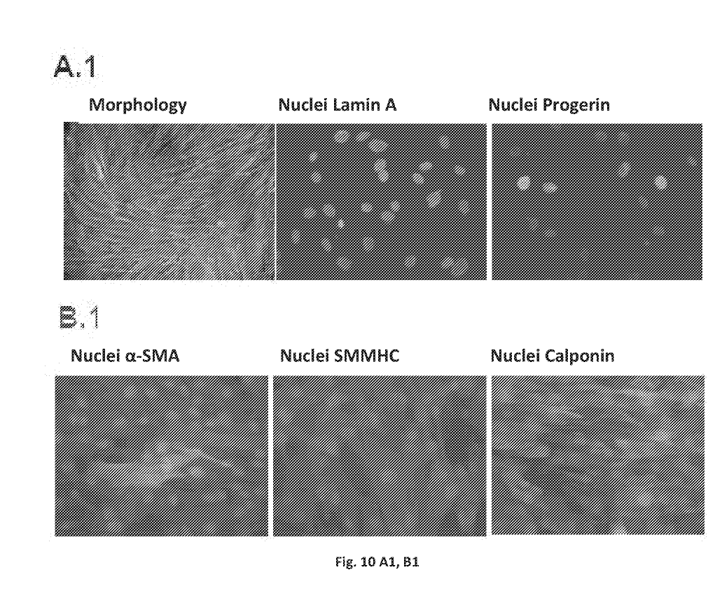

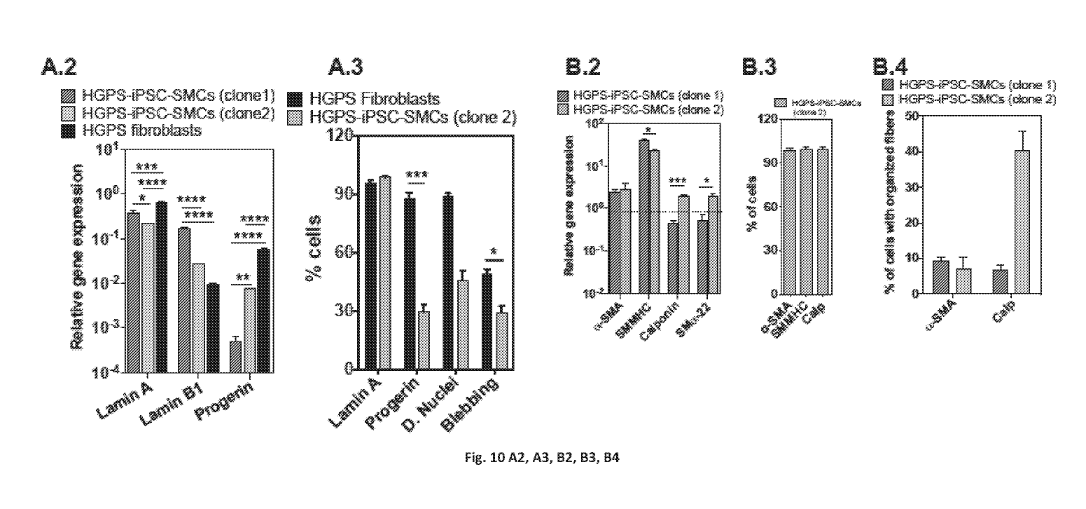

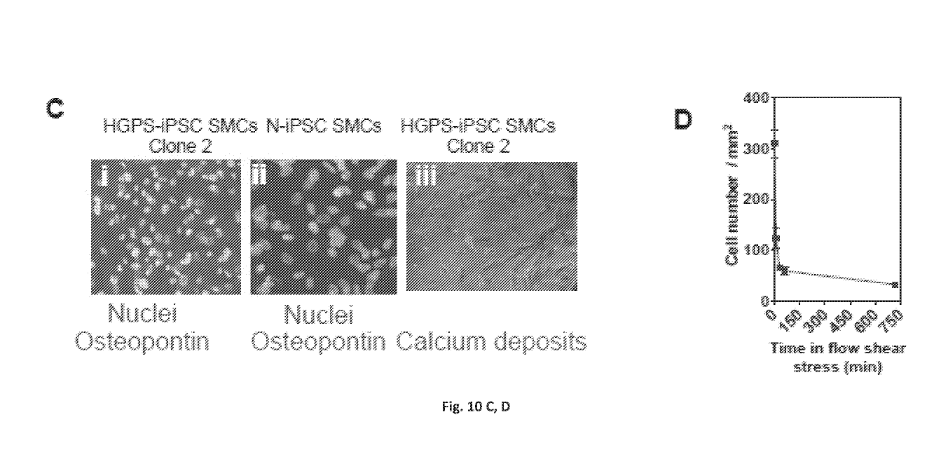

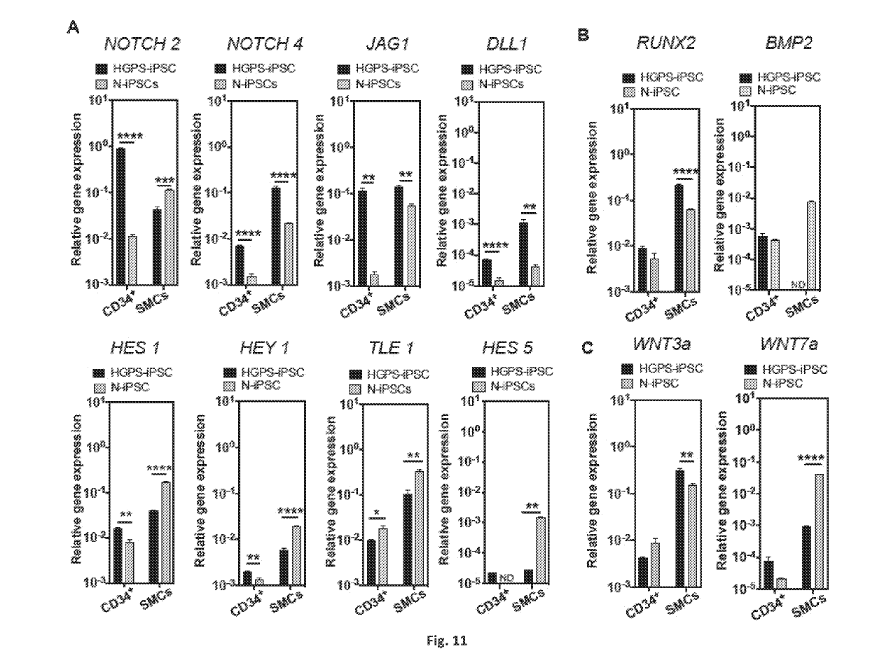

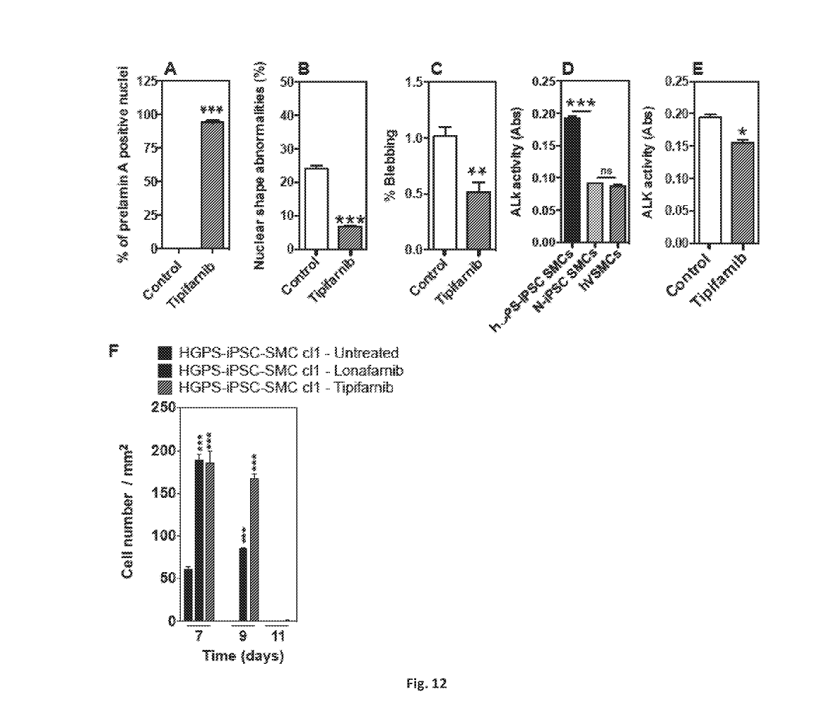

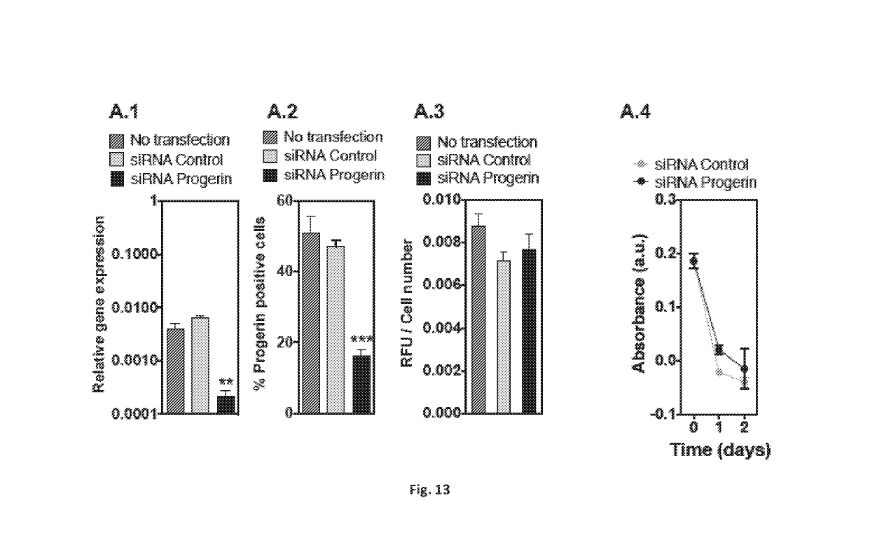

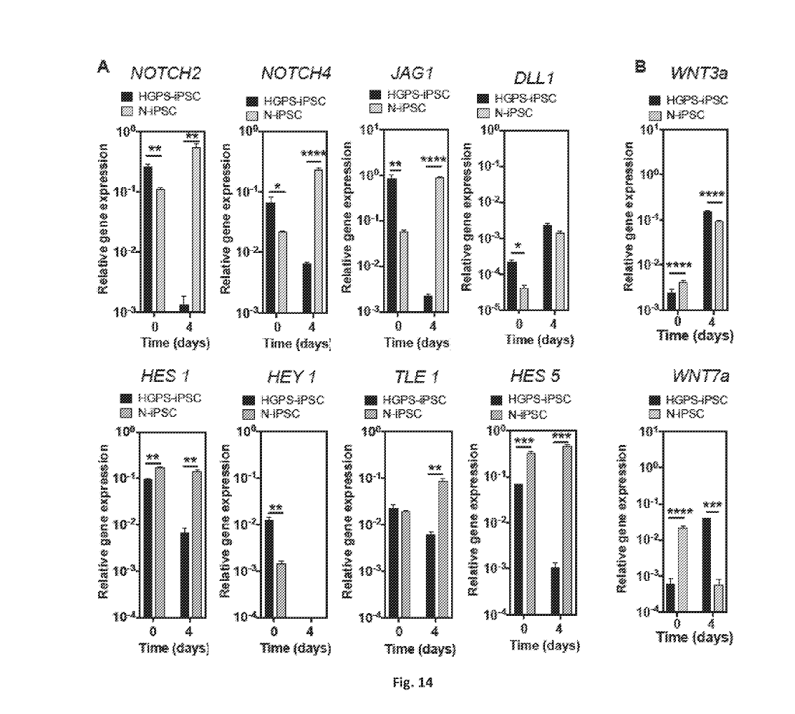

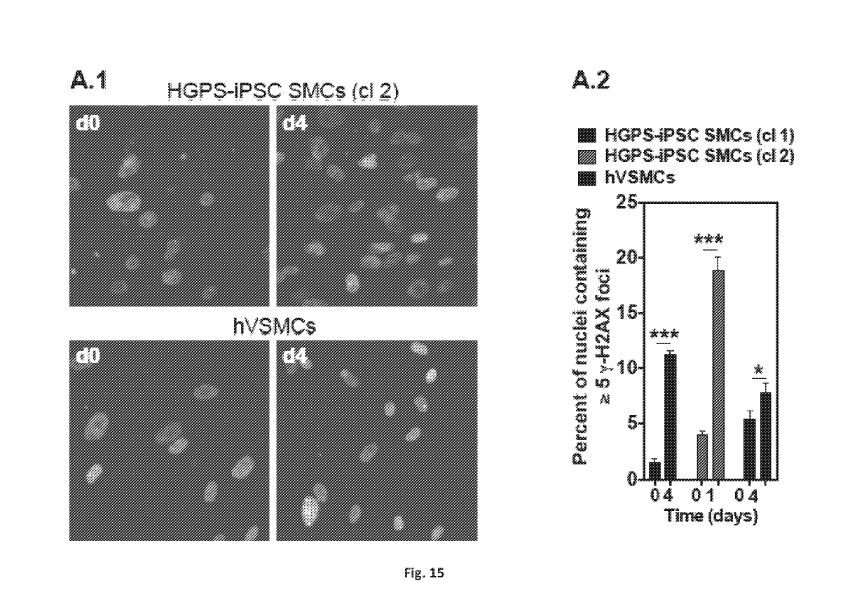

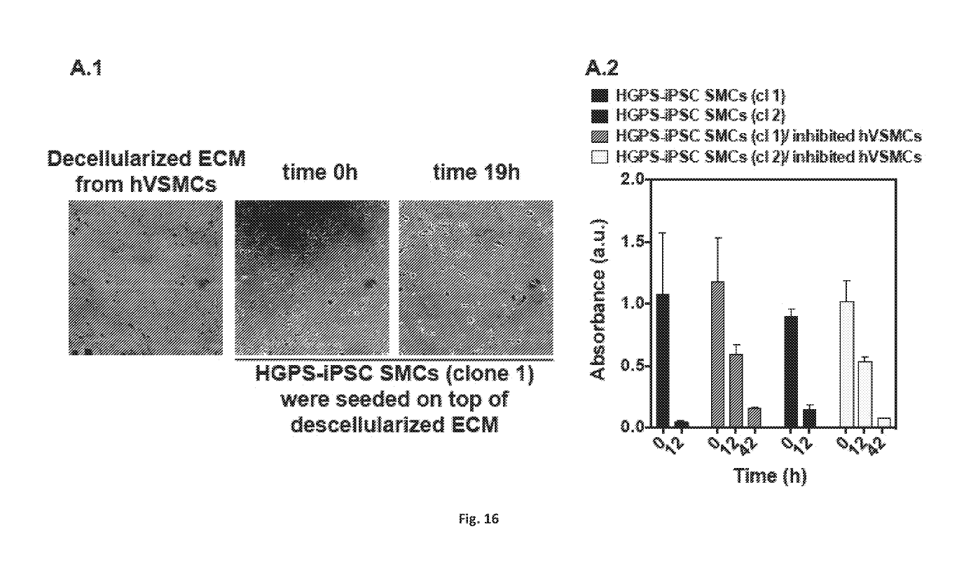

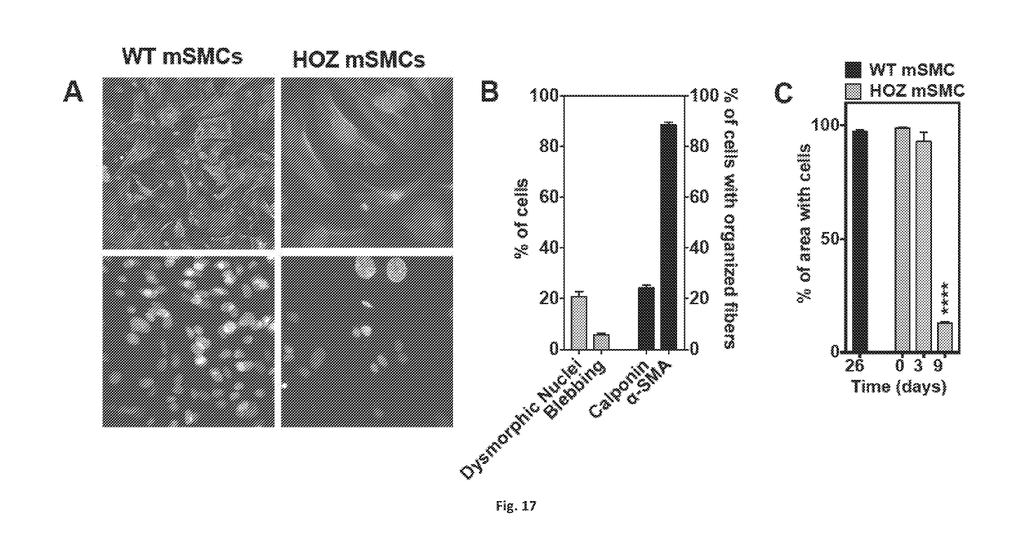

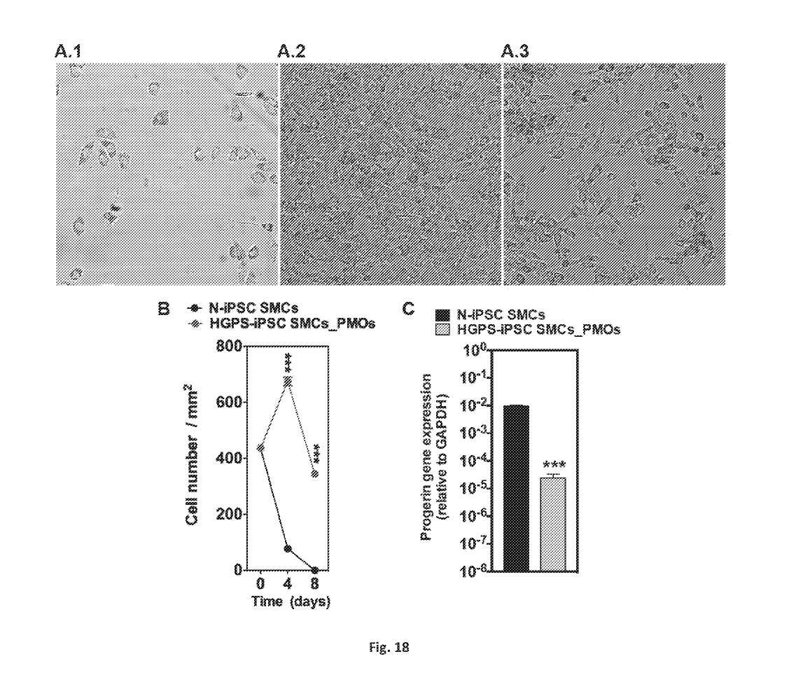

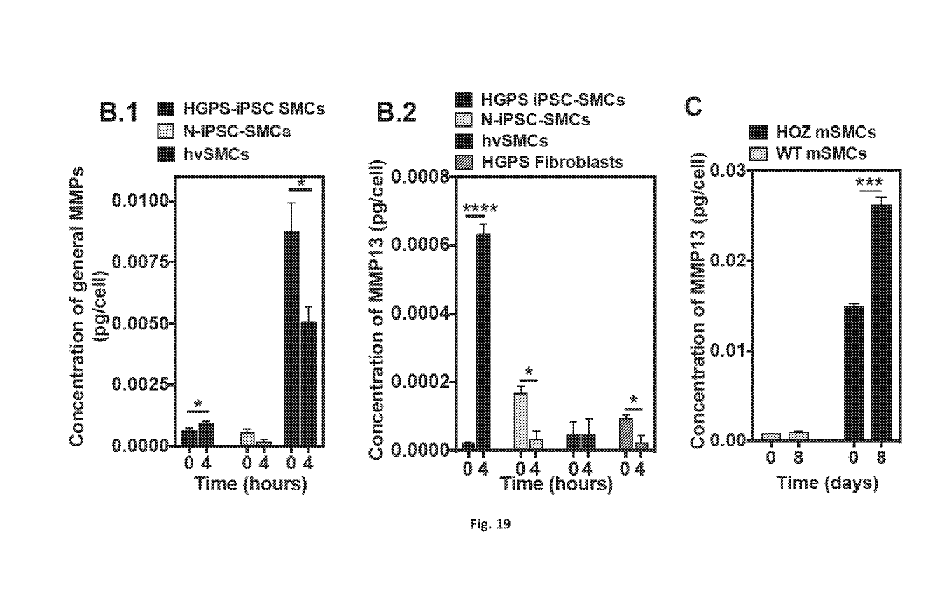

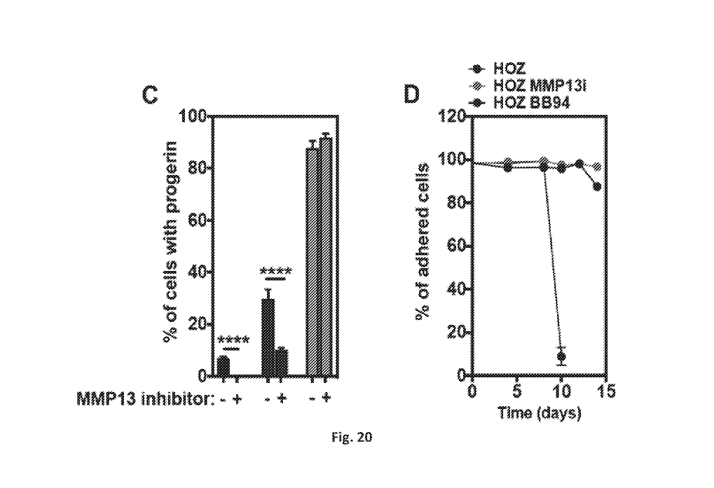

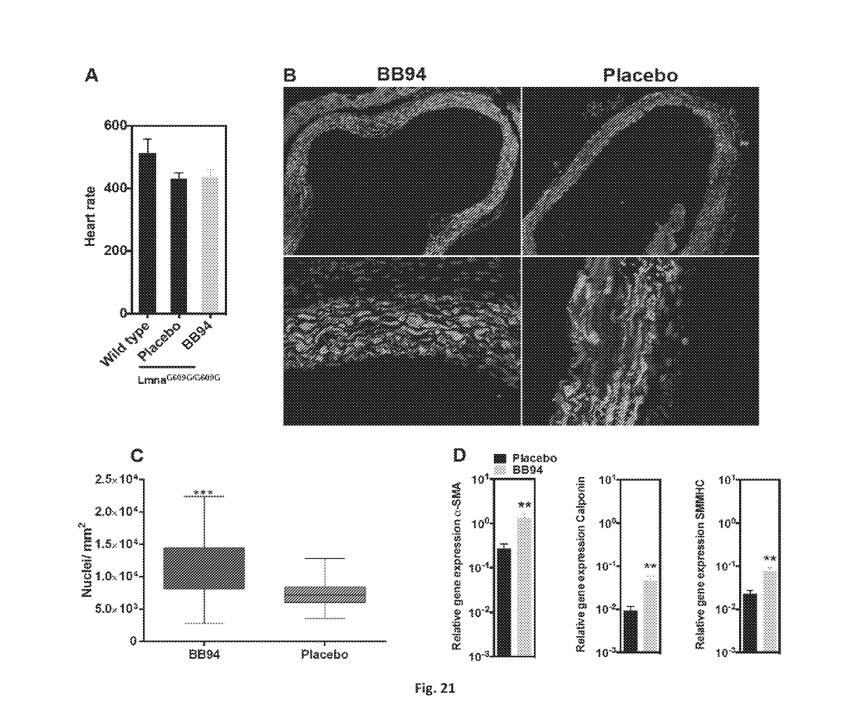

To induce the differentiation of HGPS-iPSCs or N-iPSCs into SMCs, CD34+ cells were isolated by magnetic activated cell sorting from embryoid bodies (EBs) cultured for 10 days in suspension (FIG. 1B). At this stage, HGPS-CD34+ cells already express high levels of mRNA progerin transcripts but relatively low levels of SMC mRNA transcripts as compared to N-CD34+ cells (FIG. 8). The SMC genes included: α-smooth muscle actin (α-SMA), an early marker of SMC differentiation, smooth muscle myosin heavy chain (SMMHC), a later marker in SMC differentiation; calponin (Calp) and smooth muscle α-22 (SMα-22), definitive SMC markers. The cells were then plated on gelatin-coated dishes at low density (1.5×10⁴cm²) and cultured on SMC inductive media containing PDGFBB for 4 passages (Vazao H, das Neves R P, Graos M, Ferreira L. Towards the maturation and characterization of smooth muscle cells derived from human embryonic stem cells. PLoS One. 2011;6:e17771). In case of HGPS-iPSCs, the differentiated cells express higher levels of mRNA progerin and SMC transcripts than HGPS-CD34⁺ cells (FIG. 9); however, the organization of the contractile proteins was poor and thus a further protocol step was required.

To induce maturation into SMCs, cells were cultured for further 4 passages in PDGFBB-free media (FIG. 1B). At this stage the cells were labeled as HGPS-iPSC SMCs or N-iPSC SMCs based on the phenotype, genotype and functional properties (see below). HGPS-iPSC SMCs (both clones 1 and 2) expressed lower levels of calponin and SMMHC genes than N-iPSC SMCs, as assessed by qRT-PCR (FIG. 1C.1 and FIG. 10B.2). Above 95% of both differentiated cells express α-SMA, SMMHC and calponin proteins (FIGS. 1C.2, 1E, FIG. 10B.3); however, HGPS-iPSC SMC clone 1, but not HGPS-iPSC SMC clone 2, showed relatively lower levels of individualized calponin fibers as compared to N-iPSC SMCs (≈8% versus ≈45%) (FIG. 1C.3 and FIG. 10B.4), which suggests an impairment in the maturation of HGPS-iPSCs. It has not observed heterogeneous sized calponin 1-staining inclusion bodies in the cytoplasm as reported in a previous study. Moreover, HGPS-iPSC SMCs (clones 1 and 2) express high levels of progerin gene (FIG. 1D.1 and FIG. 10A.2) and progerin protein (10 and 30% of HGPS-iPSC-SMCs clone 1 and 2, respectively, express progerin). Although these cells express lower levels of progerin protein than HGPS fibroblasts (>80% of the cells express progerin) (FIGS. 1D.2 and 1E), it should be noted that differentiated HGPS-iPSCs were cultured for 8 passages while HGPS fibroblasts were cultured for more than 24 passages. Taken together, HGPS-iPSC SMCs expressed progerin and in some cases showed signs of lower maturation than N-iPSC SMCs.

To evaluate the functionality of N-iPSC-SMCs and HGPS-iPSC-SMCs, the variation of intracellular calcium to vasoactive agents such as histamine (FIG. 1F) and angiotensin (FIG. 1F) was monitored by fluorescence. hVSMCs and human umbilical vein endothelial cells (hUVECs) were used, as positive and negative controls, respectively. Both HGPS-iPSC-SMCs and hVSMCs had similar responses after exposure to histamine and angiotensin. To examine whether HGPS-iPSC SMCs were able to contract, they were subjected to the effects of carbachol and atropine (FIG. 1G). hVSMCs and a human keratinocyte cell line (HACAT) were used as positive and negative controls, respectively. As expected, with the exception of HACAT cells, all cells were able to contract after exposure to carbachol (from 32 to 53%). Cell contraction was not significantly different from the one observed for hVSMCs. Additionally, it was verified that atropine decreased the carbachol effect, decreasing cell contraction (0.4 to 18%). Overall, our results show that HGPS-iPSC SMCs are functional as N-iPSC SMCs and somatic hVSMCs.

SMCs derived from HGPS-iPSCs share similar features observed on progerin-expressing cells. It has been shown that cell lines forced to express progerin show activation of several effectors of the NOTCH signaling pathway. Indeed, our results showed that HGPS-iPSC CD34+ cells (clone 1) had higher expression of up—(NOTCH2, NOTCH4, JAG1 and DLL1) and downstream (HES1, HES5, HEY1, TLE1) NOTCH signaling pathway genes than N-iPSC CD34⁺ cells (FIGS. 2A and 2A). Similar results were obtained for HGPS-iPSC CD34⁺ cells (clone 2) (FIG. 11). In addition, with the exception of some genes (e.g. HES1, HEY1 and TLE1 genes), the remaining ones were higher expressed on HGPS-iPSC SMCs than on N-iPSC SMCs. Because the activation of NOTCH signaling induces an osteogenic differentiation program on SMCs then it was decided to evaluate the expression of osteogenic markers such as RUNX2 and BMP-2. Previous studies have shown that BMP-2-MSX2 signaling modulates the formation of vascular calcification. Both RUNX2 and BMP2 genes expression was higher in HGPS-iPSC CD34⁺ cells (both clones) than in N-iPSC CD34⁺ cells; however, the expression profile varied on SMCs depending in the clone (FIG. 2B and FIG. 11). Similar results were obtained for Wnt3a and Wnt7a, two major Wnt agonists that mediate MSX2 effect (FIG. 2C). Taken together, our results show that NOTCH signaling pathway is activated on HGPS-iPSCs at early stages (CD34⁺ cells) before the differentiation into SMCs. This activation induces an osteogenic program as confirmed by the up-regulation of RUNX2 and BMP2.

To evaluate whether HGPS-iPSC SMCs could respond to farnesyltransferase inhibitors, as shown for other progeria cell models, cells were treated with one dose of Tipifarnib (1 μM) and after 48 h cells were characterized for the expression of prelamin A. As expected, HGPS-iPSC SMCs accumulate nuclear prelamin A (approximately 95% of the cells), as shown by immunofluorescence (FIG. 12). In addition, HGPS-iPSC SMCs show a decrease in the nuclear shape abnormalities and nuclear blebbing.

HGPS-iPSC SMCs are vulnerable to arterial shear stress—In an embodiment SMCs differentiated from N-iPSCs or HGPS-iPSCs were seeded in a microfluidic system and cultured under flow up to 7 days. Because SMCs from large arteries are the most affected blood vessels in HGPS, it was used a flow of 20 dyne/cm², typically found in arterial blood vessels (Chiu J J, Chien S. Effects of disturbed flow on vascular endothelium: Pathophysiological basis and clinical perspectives. Physiol Rev. 2011;91:327-387). N-iPSC SMCs, hVSMCs or HGPS-fibroblasts (80% of the cells express progerin) can be cultured in the microfluidic system for at least 7 days without visible loss of cell number (FIGS. 3A and 3C). In contrast, HGPS-iPSC SMCs (10% of the cells express progerin at day 0) cultured under flow conditions formed cell clumps (FIG. 3B) overtime and most of the cells detached from the substrate at day 4 as confirmed by cell number (FIG. 3C) and metabolic analyses (FIG. 3D). A second clone of HGPS-iPSC SMCs (30% of the cells express progerin at day 0) was tested and cells detached from the surface of the microfluidic system after few hours (<12 h) (FIG. 10D), confirming again the same trend as the first clone. Before cell detachment, HGPS-iPSC SMCs showed a poor proliferation as confirmed by Ki67 staining (FIG. 3E), however showed similar levels of apoptosis as compared to N-iPSC SMCs (FIG. 3F). All together, our results show that HGPS-iPSC SMCs cultured under flow conditions are vulnerable to arterial flow stress and the kinetics of cell detachment is linked to the % of progerin-expressing cells.

In an embodiment to evaluate the effect of flow shear stress on progeria and SMC expression profile on HGPS-iPSC SMCs it was performed gene expression analysis. Progerin mRNA levels did not change significantly from day 1 to day 6 in cells under flow (FIG. 3G); however, at protein level, progerin peaked at day 4, immediately before cell detachment (FIG. 3H). Nuclear abnormalities such as dysmorphic nuclei and nuclei blebbing also peaked at day 4. The inhibition of progerin by siRNA did not alter the HGPS-iPSC SMC detachment profile (FIG. 13). Moreover, HGPS-iPSC SMCs show an up-regulation of SMC markers after flow culture conditions (FIG. 3I). The most affected gene was α-SMA, which was up-regulated more than 1000 fold from day 2 to day 4. An up-regulation of SMC markers was also observed on N-iPSC SMCs; however, gene expression on these cells at day 4 was statistically lower than the one observed for HGPS-iPSC SMCs. The percentage of differentiated HGPS-iPSC SMCs with individualized calponin fibers increased from 8% at day 0 up to 15% at day 4 (FIG. 3J), which indicates maturation of HGPS-iPSC SMCs. Overall, our results indicate that HGPS-iPSC SMCs cultured on flow conditions show an up-regulation of progeria and SMC markers that peaked before cell detachment.

In an embodiment HGPS-iPSC SMCs cultured in arterial flow conditions showed an osteogenic differentiation program. To further investigate the effect of arterial shear stress on HGPS-iPSC SMCs, it was evaluated cellular expression of osteogenic markers. In contrast to N-iPSC SMCs, HGPS-iPSC SMCs showed an up-regulation in the expression of alkaline phosphatase (FIG. 4B) and osteopontin (FIG. 4C). Moreover, HGPS-iPSC SMCs and N-iPSC SMCs were also cultured in osteogenic media to enhance mineralization. A significant increase in mineralization was observed on HGPS-iPSC SMCs relatively to N-iPSC SMC (FIG. 4E). This was associated with an increase in alkaline phosphatase activity (FIG. 4D). Because SMC osteogenic differentiation proceeds through several pathways, including NOTCH, BMP, WNT and DNA damage, it was evaluated then their contribution in the osteogenic program of HGPS-iPSC SMCs. qRT-PCR analysis revealed that the mRNA levels of the osteogenic markers RUNX2 and BMP2 (FIG. 4A), as well as NOTCH effectors (FIG. 14A) decreased during HGPS-iPSC SMC culture under flow conditions. In contrast, WNT (FIG. 14B) and DNA damage markers (FIG. 15) were up-regulated suggesting that both pathways might be involved in the osteogenic program of HGPS-iPSC SMCs. It was evaluated whether the inhibition of the calcification process with sodium pyrophosphate could prevent SMC detachment. Interestingly, a significant decreased in SMC detachment was observed (FIG. 4F). Overall, our results indicate a link between progerin accumulation, osteogenesis and SMC detachment.

Microarray analysis reveals that HGPS-iPSC SMCs have significant changes in extracellular matrix (ECM) secretion and MMP expression.—To gain insights into the mechanism behind SMC detachment, it was performed microarray analyses comparing HGPS-iPSC SMCs (clone 1) at day 0 and day 4 (before cell detachment). Overall, there were 447 significantly regulated genes (2 fold changes, P<0.05), of which 234 and 213 were upregulated and downregulated, respectively. The 5 biological processes most significantly regulated were metabolic processes, cellular processes, cell communication, developmental processes and cell adhesion (FIG. 5A). From 37 genes that were 3-fold down or upregulated as compared to day 0 (P<0.001), 5 were related to ECM secretion (COL6A3—collagen type VI, alpha 3; iBSP-integrin-binding sialoprotein; BGN-biglycan; SGCG-sarcoglycan, gamma; EPPK1—epiplakin1) and 1 was related to metalloproteases (MMP13). The expression of these genes, as well as others, was further confirmed by qRT-PCR (FIG. 5B). In addition, the expression of MMP13 protein was 4-fold up-regulated in two HGPS-iPSC SMC lines (clone 1 and 2) after arterial flow conditions (FIG. 5C). The activity assessment of 459 biological pathways revealed that gonadotropin releasing hormone receptor, integrin and Wnt signaling pathways were the most affected (FIG. 5A). The expression of Wnt3a and Wnt7a genes were significantly downregulated in HGPS-iPSC SMCs after 4 days in arterial flow (FIG. 5D).

To further explore the gene array results it was evaluated whether the presence ECM secreted by hVSMCs could prevent the detachment of the HGPS-iPSC SMCs under arterial flow conditions. Thus, it was cultured HGPS-iPSC SMCs on descelullarized ECM deposited by hVSMCs or directly on top of mitotically-inactivated hVSMCs (FIG. 16). Both conditions were unable to prevent HGPS-iPSC SMC detachment (for both clones). Next it was tested whether the chemical inhibition of MMPs could prevent HGPS-iPSC SMC detachment. For that purpose it was used Batimastat (BB-94) (Wojtowicz-Praga S, Low J, Marshall J, Ness E, Dickson R, Barter J, Sale M, McCann P, Moore J, Cole A, Hawkins M J. Phase i trial of a novel matrix metalloproteinase inhibitor batimastat (bb-94) in patients with advanced cancer. Invest New Drugs. 1996;14:193-202), a broad spectrum matrix metalloprotease inhibitor MMP-1, MMP-2, MMP-9, MMP-7 and MMP-3 (IC50≦20 nM), and a specific MMP-13 inhibitor, pyrimidine-4,6-dicarboxylic acid, bis-(4-fluoro-3-methyl-benzylamide) (IC50=8 nM) (Engel C K, Pirard B, Schimanski S, Kirsch R, Habermann J, Klingler O, Schlotte V, Weithmann K U, Wendt K U. Structural basis for the highly selective inhibition of mmp-13. Chem Biol. 2005;12:181-189), have been used. Remarkably, both inhibitors decreased significantly the detachment of both clones of HGPS-iPSC SMCs cultured under arterial flow conditions (up to 12 days) (FIG. 5E). The effect of BB-94 and MMP-13 inhibitor was compared to Tipifarnib (1 μM) and Lonafarnib (20 μM) in HGPS-iPSC SMC clone 1. Both farnesyltransferase inhibitors were able to decrease cellular detachment, maintaining cells under flow conditions up to 9 days (FIG. 12). This shows that MMP inhibitors are more efficient in preventing cell detachment than farnesyltransferase inhibitors. Finally, MMP-13 inhibitor used under flow conditions was also able to decrease the percentage of progerin positive cells (FIG. 5G) and alkaline phosphatase activity levels.

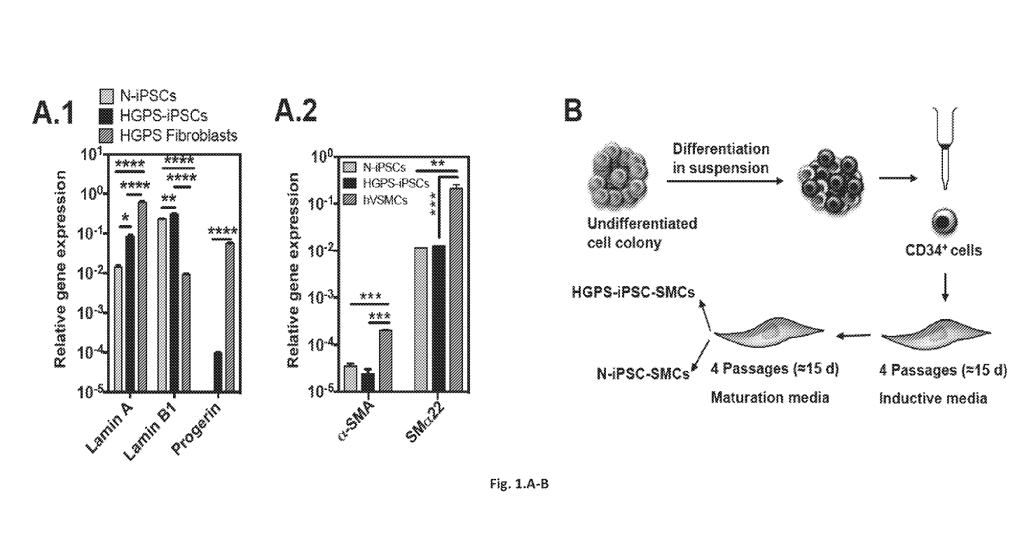

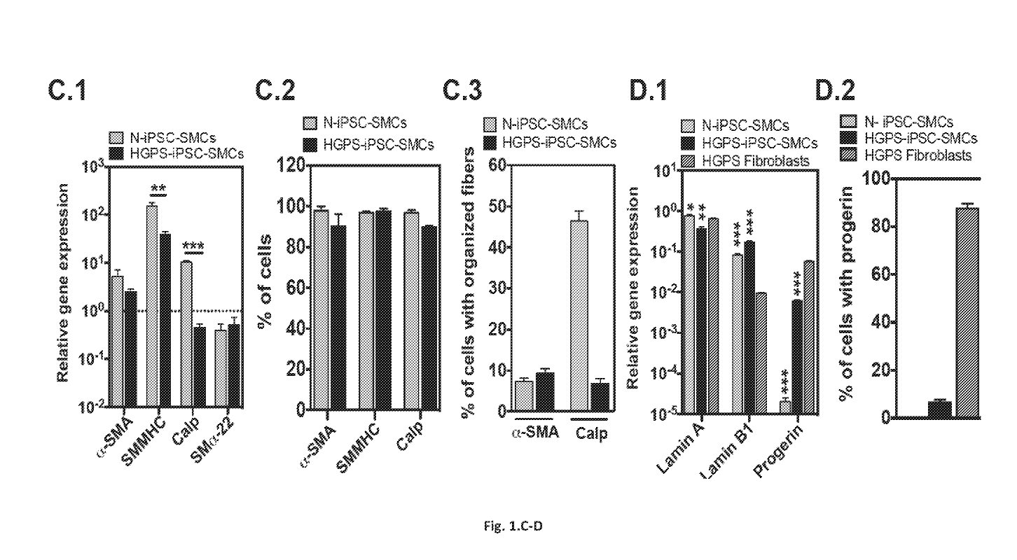

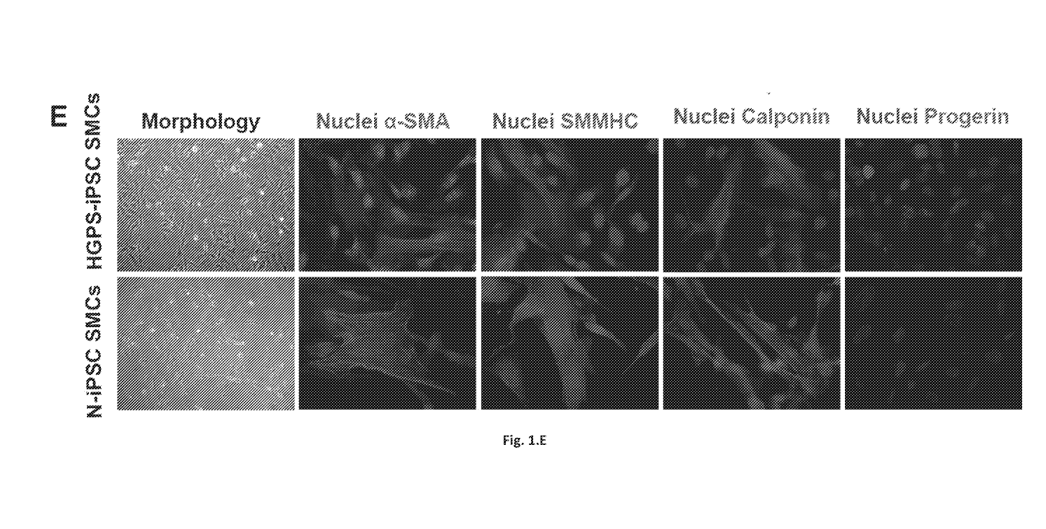

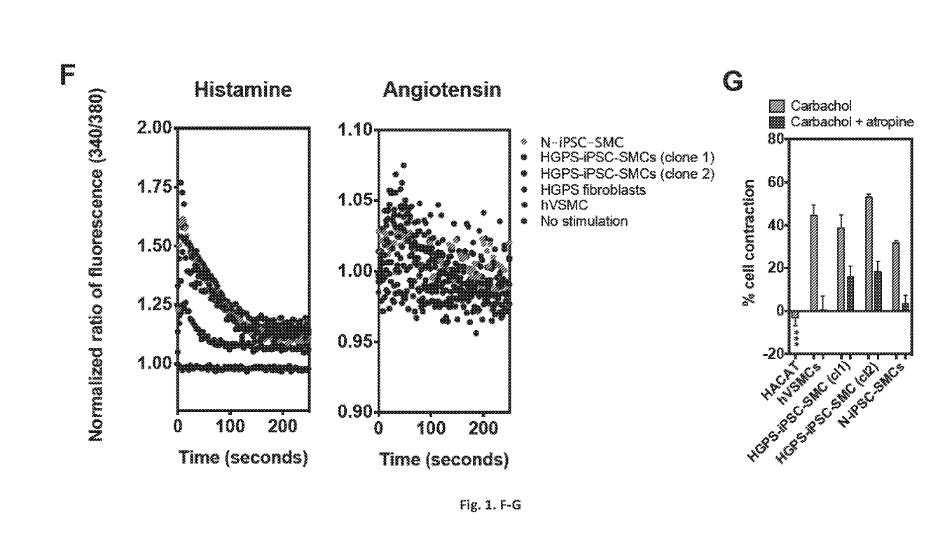

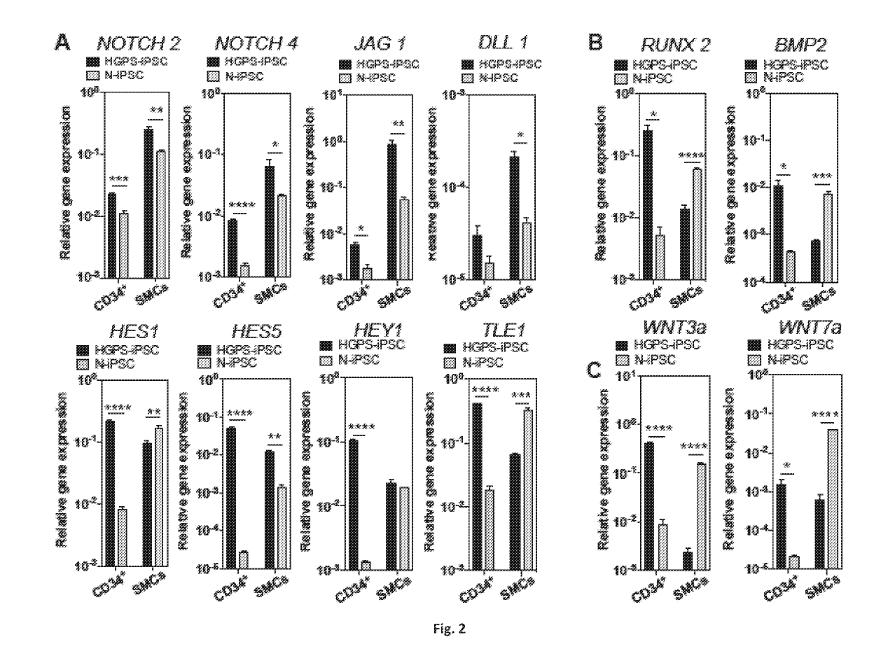

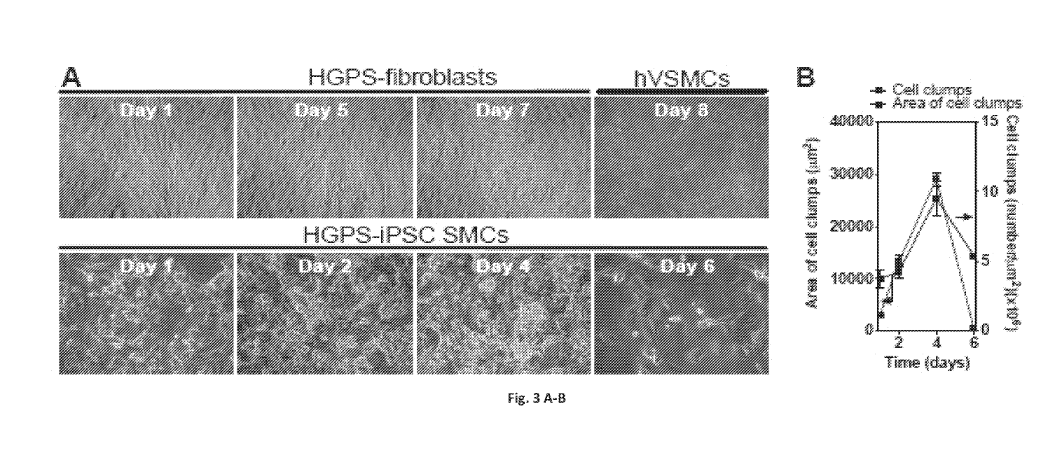

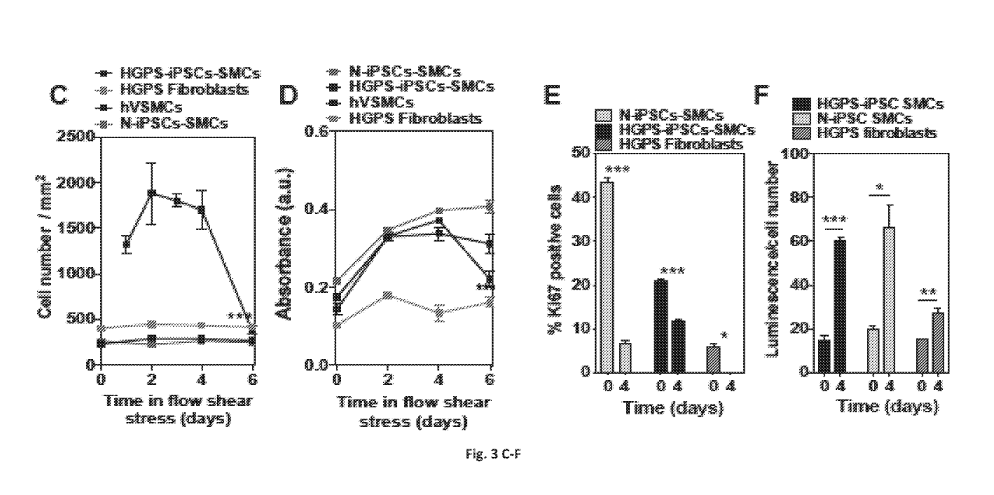

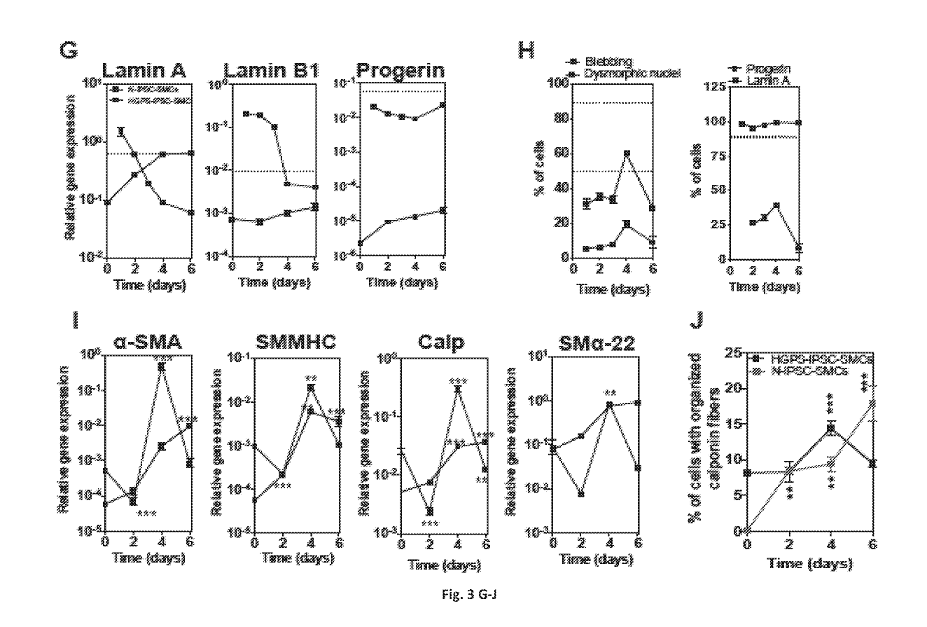

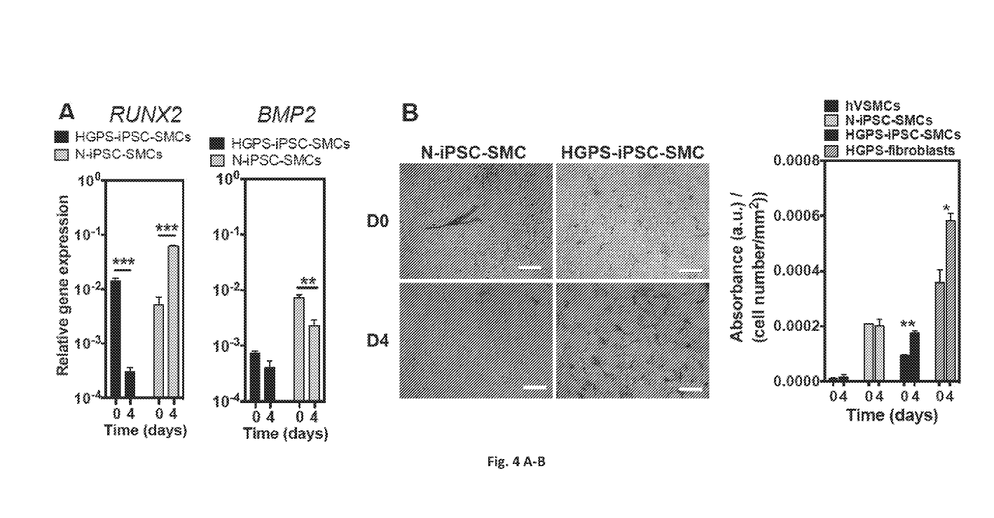

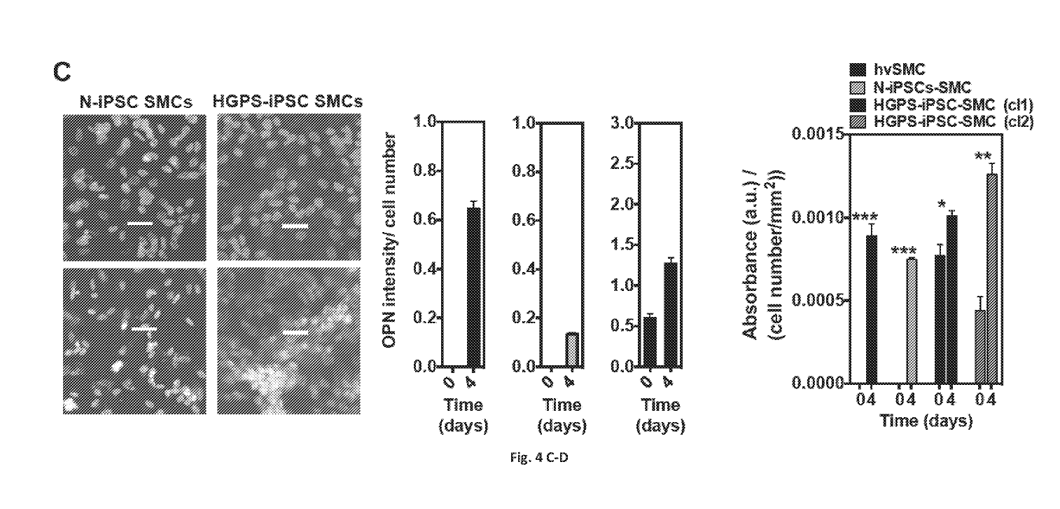

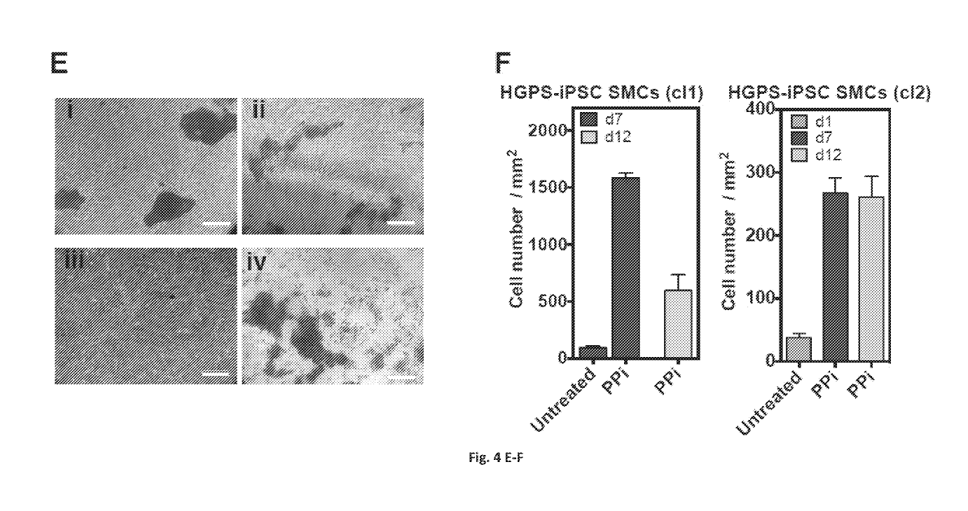

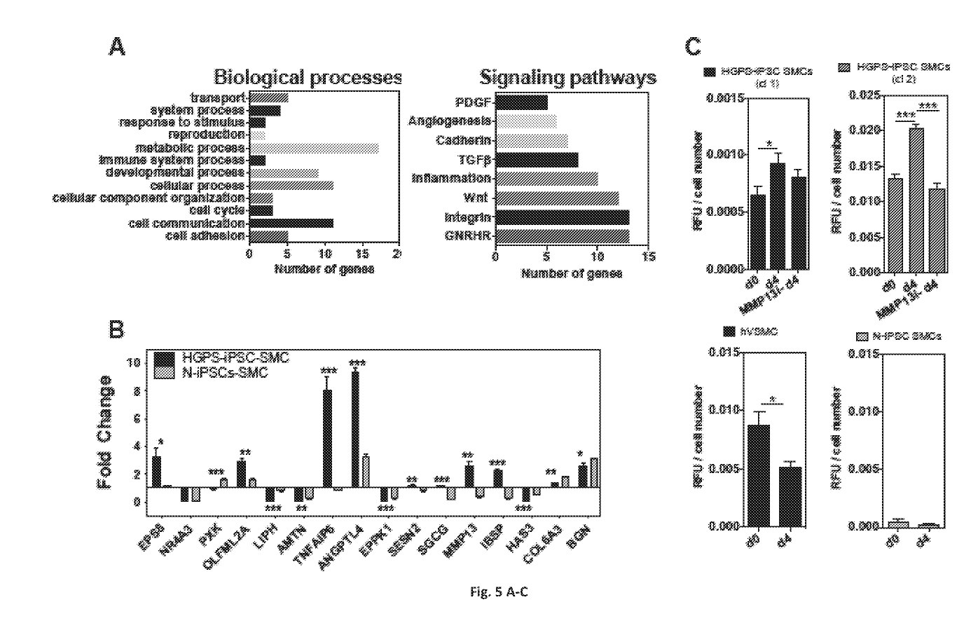

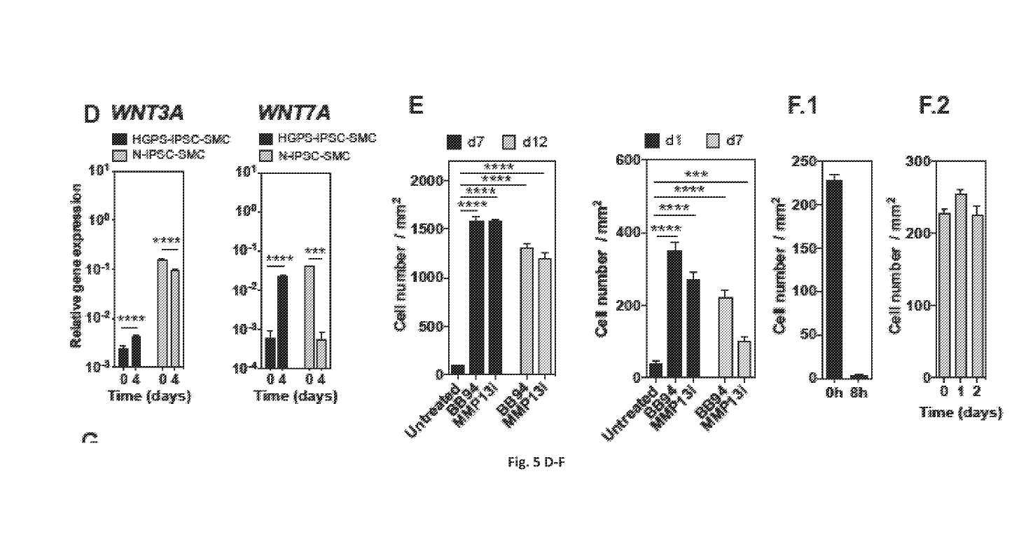

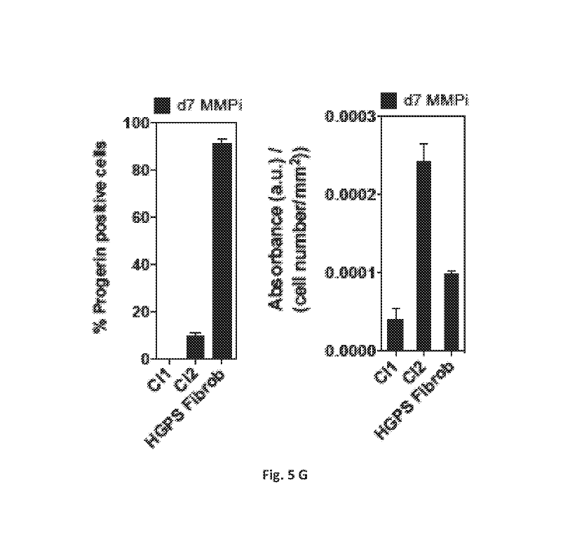

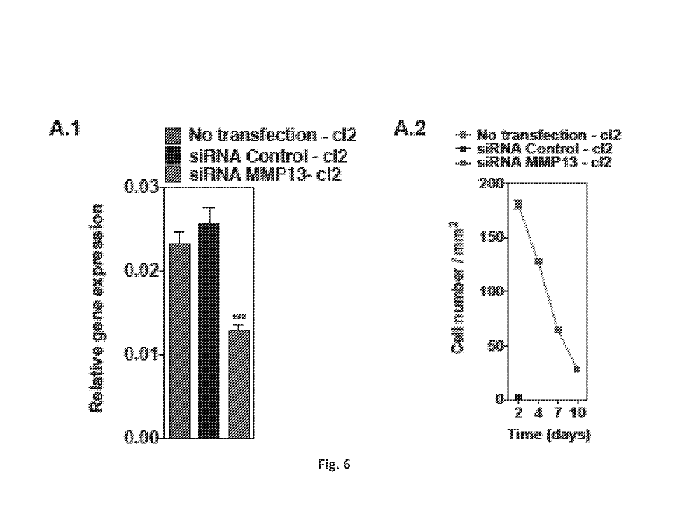

To confirm these results, HGPS-iPSC SMCs clone 2 were knockdown for MMP13 by siRNA and cultured under arterial flow conditions for 10 days (FIG. 6). In contrast to untreated cells, cells knock down for MMP13 have low propensity to detach from the substrate. To further confirm these results, HGPS-iPSC SMC conditioned media (collected after 4 days of culture under arterial flow conditions) was exposed to N-iPSC SMCs. In these conditions, N-iPSC SMCs detached from the substrate after 1 day of culture showing a link between cell detachment and MMPs (FIG. 5F).

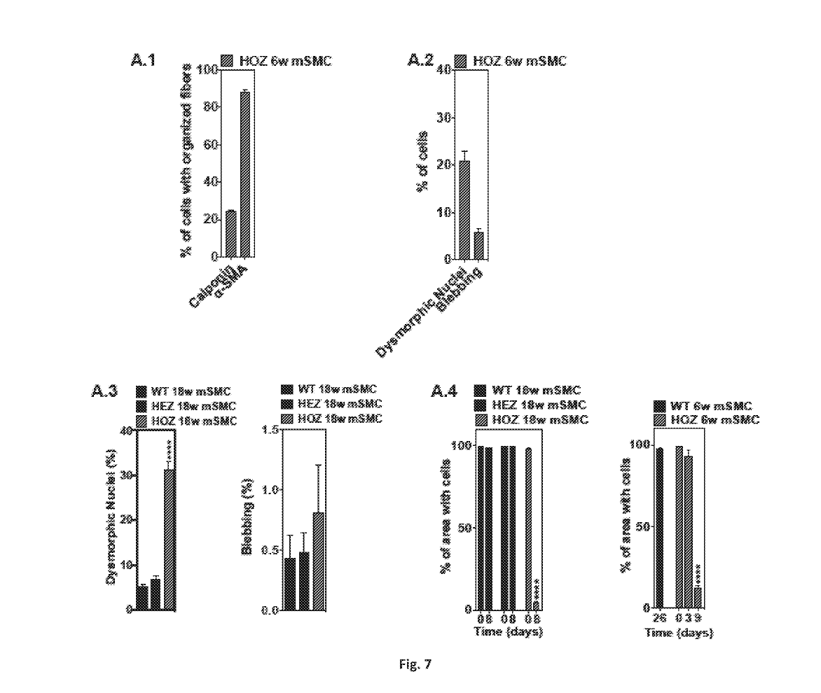

Progeria mouse SMC present a similar profile as HGPS-iPSC SMC in terms of cell detachment. It has been shown that wild-type mouse Lmna gene with a mutant allele that carried the c.1827C>T;p.Gly609Gly mutation recapitulate most of the described alterations associated with HGPS, including the loss of vSMC. Take this into account it was isolated SMC from wild-type mice (WT mSMC), heterozygous LmnaG609G/+ mice (HEZ mSMC) and homozygous Lmna G609G/G609G (HOZ mSMC). These cells were isolated at 6 weeks and 18 weeks and they were characterized in terms of percentage of dysmorphic nuclei and blebbing and SMC fibers (FIG. 7). As expected HOZ mSMC presented higher levels of dysmorphic nuclei and blebbing comparing to HEZ mSMC and WT mSMC, and mSMC isolated at week 18 have high level of dysmorphic nuclei as compared with mSMC isolated at week 6. In addition, mSMC showed high percentage of organized SMC fibers. To confirm the human SMC results it was cultured mSMC under flow shear stress conditions (120 dyne/cm²) and it was verified that WT mSMC were able to be cultured under flow conditions up to 26 days without loss of cells. On the other hand, HOZ mSMC detached from the substrate after 8/9 days. These results confirm that mSMC are vulnerable to flow shear stress and are in agreement with HGPS-iPSC SMC results.

Progerin accumulation during the differentiation of iPSCs activates NOTCH signaling—In an embodiment, it was showed that the differentiation of HGPS-iPSCs induces the activation of NOTCH signaling pathway. This is observed at the isolation of CD34⁺ cells and after their differentiation into SMCs (for both clones of HGPS-iPSCs). Our results indicate that progerin activates major upstream (NOTCH2, NOTCH4, JAG1, DLL1) and downstream (HES1, HES5, HEY1 and TLE1) effectors on CD34⁺ cells; however the expression of downstream effectors is downregulated afterwards during their differentiation into SMCs. Previously, it was demonstrated that the induction of progerin expression on somatic cells increased the pool of SKIP molecules which in turn activated major downstream effectors of the NOTCH signaling pathway including HES1 (Hairy and enhancer of split1), HES5, HEY1 and TLE1 (Scaffidi P, Misteli T. Lamin a-dependent misregulation of adult stem cells associated with accelerated ageing. Nat Cell Biol. 2008;10:452-459). Curiously, the activation of NOTCH effectors was not mediated by changes in expression levels of upstream components of the pathway. These results are not in line with the present results that show a clearly activation of both type of effectors. The differences observed might account for the differences in the developmental stage of both type of cells.

Progerin expression during the differentiation of iPSCs activates an osteogenic differentiation program—In an embodiment, it was showed that progerin expression activated initially the expression of osteogenic markers Runx2 and BMP2 on CD34⁺ cells. HGPS-iPSC SMCs with low progerin protein expression (ca. 10%) show low levels of alkaline phosphatase, osteopontin and mineralization. However, the culture of HGPS-SMCs under flow conditions induced the expression of alkaline phosphatase, osteopontin and mineralization as assessed by alizarin red staining. This osteogenic program occurred during up-regulation of progerin protein in the cells. Moreover, HGPS-iPSC with moderate levels of progerin protein expression (ca. 30%) showed high levels of osteopontin and alkaline phosphatase. Therefore, our results suggest a link between accumulation of progerin on SMCs and their osteogenic differentiation. SMC osteogenic differentiation may occur through several pathways including NOTCH, BMP, WNT and DNA damage pathways. Our results show that the osteogenic conversion of SMCs is not mediated by the activation of NOTCH or BMP signaling (FIG. 14 and FIG. 4A). According to our results, it is likely that the osteogenic program is induced by DNA damage or WNT signaling.

SMCs derived from HGPS-iPSCs are vulnerable to arterial flow—Our results indicate that SMCs derived from HGPS-iPSCs but not from N-iPSCs are sensitive to arterial flow shear stress and detach from the substrate. The kinetics of HGPS-iPSC SMC detachment is linked to the level of progerin accumulated in the cells, i.e, faster for cells that accumulate higher levels of progerin. To the best of our knowledge this is the first in vitro system showing SMC loss under arterial flow conditions. A previous report has demonstrated the sensitivity of HGPS fibroblasts to mechanical strain confirmed by a decreased viability and increased apoptosis under repetitive mechanical strain. Yet, the effect of mechanical strain is cell-dependent as, it was show in the current study. For example, HGPS fibroblasts with high levels of progerin accumulation (>80% of the cells have accumulation of progerin) and cultured under arterial flow conditions do not detach from the cell culture substrate.

Several mechanisms may account for the increased mechanical sensitivity of HGPS SMCs, including nuclear stiffness, calcification, apoptosis, ECM remodelling, among others. It was shown that the nuclei in HGPS cells become progressively stiffer with increasing passage; however, experimental data showed that the increased mechanical sensitivity of HGPS cells is unrelated to changes in nuclear stiffness. It is also known that aortas and aortic valves of HGPS patients are excessively calcified and arteries from old transgenic mice carrying HGPS mutation accumulate calcium deposits that are absent in age-matched controls. Therefore, SMC mechanical sensitivity may be also linked to its excessive calcification, and indeed our results show a relation between calcification and SMC loss (FIG. 4F). Our results indicate that the enhanced sensitivity of SMCs to mechanical strain is linked to dynamics in the cellular deposition of ECM and remodelling by MMPs. According to our microarray results and chemical inhibition assays, it was show that the MMPs are key players in SMC depletion. In an embodiment, it was show that the inhibition of MMP-13 by both chemically and genetically (siRNA) means reduced significantly cell depletion and reduced also the percentage of progerin positive cells and alkaline phosphatase levels. Previous studies have shown that the expression of matrix metalloproteinase 3 (MMP-3) was down-regulated in HGPS primary human dermal fibroblasts which favoured matrix deposition as observed in vascular vessels of HGPS patients. However, a link between SMC loss and MMP expression was never documented.

Cell culture—In an embodiment, HGPS fibroblasts line AG06917 (Coriell cell repositories) was cultured in DMEM (Sigma) supplemented with fetal bovine serum (FBS, 20%, v/v, Gibco), sodium pyruvate (Sigma, 1 mM) and penicillin-streptomycin (50 U/mL:50 mg/mL). Cell cultures were maintained at 37° C., 5% CO₂ in a humidified atmosphere, with media changed every 2 days.

hvSMCs (Lonza, CC-2579) were cultured in Smooth Muscle Growth Medium-2 (SmGM-2) medium (Lonza CC-3182) from passage 3 to passage 7. Cell cultures were maintained at 37° C., 5% CO₂ in a humidified atmosphere, with media changed every 2 days.

HACAT (human immortalized keratinocyte cell line) were cultured in DMEM (Sigma) supplemented with FBS (10%, v/v, Gibco) and penicillin-streptomycin (50 U/mL:50 mg/mL). Cell cultures were maintained at 37° C., 5% CO₂ in a humidified atmosphere, with media changed every 2 days.

iPSCs culture and embryoid body (EB) formation—In an embodiment, HGPS-iPSCs clone 1 (passages 43-51); HGPS-iPSCs clone 2 (passages 35-42), CB-iPSCs (passages 35-40) and N-iPSCs (passages 30-35) were maintained on mitotically inactivated mouse embryonic fibroblast (MEF) feeder layer, as previously described. To induce embryoid bodies (EBs) formation, the iPSCs were treated with collagenase IV (1 mg/mL, Gibco) for 1 h and then transferred (2:1) to low attachment plates (Corning) containing 10 mL of differentiation medium (80% KO-DMEM (Life Technologies), 20% fetal bovine serum (FBS, Invitrogen), 0.5% L-glutamine (Life Technologies), 0.2% 6-mercaptoethanol (Sigma), 1% non-essential amino acids (Invitrogen) and 50 U/mL:50 mg/mL penicillin-streptomycin solution (Lonza)). EBs were cultured for 10 days at 37° C., 5% CO2 in a humidified atmosphere, with media changes every 2 days.

Isolation and differentiation of CD34⁺ cells. CD34⁺ cells were isolated from EBs at day 10 (Ferreira L S, Gerecht S, Shieh H F, Watson N, Rupnick M A, Dallabrida S M, Vunjak-Novakovic G, Langer R. Vascular progenitor cells isolated from human embryonic stem cells give rise to endothelial and smooth muscle like cells and form vascular networks in vivo. Circ Res. 2007;101:286-294). The percentage of CD34⁺ cells in EBs was between 0.4 and 1.5%. Isolated cells were grown on 24-well plates (˜3×104 cells/cm²) coated with 0.1% gelatin in the presence of endothelial growth medium-2 (EGM-2, Lonza) supplemented with PDGFBB (50 ng/mL, Prepotech). After 4 passages, the medium was replaced by Smooth Muscle Growth Medium-2 (SmGM-2) (Lonza CC-3182) (maturation medium), for additional 4 passages. hVSMCs (Lonza) were used as controls for the differentiation studies. Cell cultures were maintained at 37° C., 5% CO₂ in a humidified atmosphere, with media changed every 2 days.

Intracellular Ca²⁺ variation measurements—In an embodiment measurement of intracellular Ca²⁺ were performed as described before. Briefly, hVSMCs or HGPS-iPSC SMCs or N-iPSC SMCs were loaded with a Fura-2 calcium fluorescent indicator solution formed by acetoxymethyl (AM) derivative FURA-2/AM (5 mM, 1 mM in DMSO, Invitrogen), Pluronic F-127 (0.06%, w/v, Sigma) and M199 basal medium (Sigma) (35 μL/well, not supplemented with serum nor antibiotics), for 1 h at 37° C. in 5% CO₂ and 90% humidity. Cells were then stimulated with histamine (100 μM, Sigma) or angiotensin (10-5 M, Calbiochem), by adding 1 mL of a stock solution. hVSMCs and HGPS fibroblasts were used as controls.

Contractility assays. In an embodiment, measurement of SMCs contractility was assessed as described before (Vazao H, das Neves R P, Graos M, Ferreira L. Towards the maturation and characterization of smooth muscle cells derived from human embryonic stem cells. PLoS One. 2011;6:e17771). HGPS-iPSC SMCs and N-iPSC SMCs cultured for 8 passages were washed with DMEM (Sigma) and contraction was induced by incubating these cells with 10-5 M carbachol (AlphaAesar) in DMEM (Sigma) medium for 30 min. Contraction was calculated as the difference in cell area (assessed by microscopy) between time zero and 30 min. In a distinct experiment, cell relaxation was induced by incubation with atropine (10-4 M, AlfaAesar) in DMEM (Sigma) for 1 h followed by contraction with carbachol (10-5 M, AlphaAesar). Contraction was calculated as before. hVSMCs and HACAT were used as positive and negative controls, respectively.

Quantitative reverse transcription-polymerase chain reaction (qRT-PCR) analysis—In an embodiment, messenger RNA levels from experimental groups were quantified using a Power SYBR® Green Cells-to-CT™ Kit (Applied Biosystems). All genes were measured using SYBR Green technology, with the exception of Progerin. Progerin-specific ragman primer and probe was customized and the results were normalized to glyceraldehyde 3-phosphate dehydrogenase (GAPDH, VIC®/MGB Probe, Primer Limited) (Applied Biosystems). qRT-PCR analyses were performed using a ABI PRISM 7500 Fast System (Applied Biosystems) run for 45 cycles. Quantification of target genes was performed relative to GAPDH gene according to the equation: 2[−(Ct sample −Ct GADPH)]. The mean minimal cycle threshold values (Ct) were calculated from quadruplicate reactions. The list of the primers can be found in Supplemental information.

Immunofluorescence analysis—In an embodiment, cells were washed with PBS, fixed with 4% paraformaldehyde (Electron Microscopy Sciences) for 15 min at room temperature and washed again with PBS. Cells were blocked with 1% (w/v) BSA and stained for 1 h with anti-human primary antibodies specific for smooth muscle α-actin (α-SMA, 1A4, Dako), smooth muscle myosin heavy chain (SMMHC, SMMS-1, Dako), calponin (CALP, Calponin1, Santa Cruz Biotec), Lamin A/C (H-110, Santa Cruz Biotec), Progerin (13A3DD4, Santa Cruz Biotec), osteopontin (AKm2A1, Santa Cruz Biotec), Ki-67 (Clone MIB-1, Dako) and H2AX (pS139, BD Pharmingen). In each immunofluorescence experiment, an isotype matched IgG control was used. Binding of primary antibodies to specific cells was detected with anti-mouse IgG Cy3 (Sigma) or anti-rabbit IgG Cy3 (JacksonImmunoResearch). Cell nuclei were stained with 4′, 6′-diamidino-2-phenylindole (DAPI) (Sigma) and the slides examined with a regular (Zeiss) or high-content fluorescence microscope (IN Cell 2200, GE Healthcare). Image) software was used to quantify the overall intensity of each image, which was then normalized for cell number.

Cell culture under arterial flow—In an embodiment a suspension of HGPS-iPSC SMCs (clone 1), HGPS-iPSC SMCs (clone 2), N-iPSC SMCs, hVSMCs or HGPS fibroblasts between 5×104 and 1.3×105 cells/cm2 was applied to the entry port of an IBIDI channel (μ-Slide I 0,4 Luer, or μ-Slide VI 0.4 Luer, IBIDI) and allowed to flow inside by capillary force. After 4 h, a confluent cell layer was formed, which was then perfused with SmGM-2 medium at physiological flow rate (20 dyne/cm²). Unless specified, all tests were performed at day 0 and after 4 days on flow culture conditions. Cell number and cell clumps were determined on slides stained with DAPI (20×) and normalized by image area (0.3524 mm²). Cell clumps areas were evaluated by Image J software.

Cell viability—In an embodiment cell viability was assessed by a cell permeable resazurin-based solution, PrestoBlue™ (Life Technologies). PrestoBlue™ Reagent was added directly to cells in culture medium (1:10), incubated for 2 h, and the absorbance's at 570 and 600 nm monitored by a plate reader (BioTek). The absorbance values at 570 nm were then normalized by the absorbance values at 600 nm.

Caspase-9 activity—In an embodiment Caspase 9, a key initiator of the intrinsic apoptotic pathway of mammalian cells, was measured by a Caspase-Glo® 9 Assay (Promega). Caspase-Glo® 9 Reagent (100 μL) was added to each well of a white-walled 96-well plate containing culture medium (100 μL) without cells (blank) or with cells (sample). The mix was incubated at room temperature for 30 min, after which the luminescence was measured in a plate-reading luminometer (Lumistar). Luminescence values were then normalized by the number of cells per well.

Alkaline phosphatase activity—In an embodiment, alkaline phosphatase activity was assessed either by a colorimetric substrate, 1-Step pNPP (Thermo Scientific), or SigmaFast 5-Bromo-4-chloro-3-indolyl phosphate/Nitro-blue tetrazolium (BCIP/NBT) (Sigma-Aldrich). In the case of 1-Step pNPP substrate, cells were fixed with ethanol 95% (v/v) during 15 min, washed with PBS (Sigma), and finally stained with 1-Step pNPP reagent. The mix was incubated at 37° C. during 30 min and the absorbance was monitored at 405 nm using a plate reader (BioTek). Results were normalized by cell number per mm2. In the case of SigmaFast substrate, cells were fixed with ethanol 95% (v/v) during 15 min, washed with PBS (Sigma), and then stained with the SigmaFast reagent (one tablet was dissolved in 10 mL of distilled water) for 10-15 min at 37° C. Cells were washed three times with distilled water and observed under an optical microscope.

Calcification measurements by alizarin red staining—In an embodiment, cells were washed with PBS, fixed with 10% (v/v) formaldehyde (Sigma) during 15 min at room temperature and washed three times (5-10 min each) with an excess of distilled water. Alizarin red solution (40 mM, pH 4.1, Sigma) was added to the fixed cells and incubated at room temperature for at least 20 min with gentle shaking. The excess dye was removed, washed four times with deionized H₂O, and cells observed by an optical microscope.

Loss of function studies—In an embodiment, in case of MMP inhibition studies, a suspension of HGPS-iPSC SMCs (clone 1) or HGPS-iPSC SMCs (clone 2) in SmGM2 medium was seeded in each IBIDI channel. Four hours after seeding, cells were either treated with SmGM-2 medium containing a MMP13 inhibitor (pyrimidine-4,6-dicarboxylic acid, bis-(4-fluoro-3-methyl-benzylamide)) (8 nM, Calbiochem, Merk Millipore), or a broad spectrum MMP inhibitor (20 nM, batimastat, BB-94, Selleckchem). In case of calcification inhibition studies, cells were treated with SmGM-2 medium containing sodium pyrophosphate (0.9 mM, Sigma), and in case of farnesyltrasferase inhibitor studies, cells were treated with SmGM-2 medium containing Tipifarnib (1 μM) or Lonafarnib (20 μM, Absouce Diagnostics). In MMP, calcification and farnesyltransferase inhibition studies, cells were perfused with medium containing the drug at a flow rate of 20 dyne/cm². Medium was changed every 7 days. Cell number and viability (Presto Blue assay) was monitored overtime. In case of MMP-13 inhibition studies, MMP activity was assessed at day 4, and the percentage of progerin positive cells and the expression of phosphatase alkaline were evaluated at day 7.

In case of siRNA studies, lipofectamine RNAiMAX (in DMEM, Life Technologies) was added to siRNA progerin (Sigma) or siRNA control (Izasa) (240 nM, in DMEM) in a ratio of 1:1. The complexation of siRNAs with lipofectamine was allowed to proceed for 40 min at room temperature. The complexes were then added to HGPS-iPSC SMCs (clone 1) or HGPS-iPSC SMCs (clone 2) cultured in SmGM-2 medium in a ratio of 1:3. The culture media was changed after 72 h. For siRNA MMP-13 (Santa Cruz Biotechnology—https://www.scbt.com/pt/datasheet-41559-mmp-13-sirna-h.html) studies the procedure was the same but it was used a final concentration of 100 nM and the culture media was changed after 4 h.

MMP activity—In an embodiment, MMP activity was quantified on cell extracts by a fluorometric red assay kit (Abcam). Cell extracts were obtained by incubating the cells with Triton X-100 (0.5%, v/v, in PBS, Sigma) for approximately 15 min, the cells centrifuge and the supernatant collected. Part of cell extract (25 μL) was added to 4-aminophenylmercuric acetate (APMA, 25 μL, 2 mM) and incubated for 40 min at 37° C. Then, a MMP red substrate (50 μL) was added to the mixture and the fluorescence intensity measured in a fluorometer (Ex/Em=540/590 nm) after 1 h, at room temperature.

Decellularization of the extracellular matrixes—In an embodiment, hVSMCs were cultured under flow conditions for 4 days in a IBIDI channel coated with fibronectin (50 μg/mL, Calbiochem). Cells were then washed with PBS and treated with PBS supplemented with ammonium hydroxide (20 mM) and Triton X-100 (0.5%, v/v) for 5 min at 37° C. to disrupt lipid-lipid and lipid-protein interactions. The resulting ECM layers were washed with an excess of PBS three times. Then, a suspension of HGPS-iPSC SMCs (clone 1) in SmGM-2 medium was seeded on top of decellularized ECM and after 4 h medium was flowed (20 dyne/cm²).

Microarray analyses—In an embodiment, the analyses were performed on HGPS-iPSC SMCs (clone 1) at day 0 or cultured for 4 days in arterial flow conditions. In both cases, HGPS-iPSC SMCs (clone 1) were homogenized in Trizol reagent (Life Technologies) and the total amount of RNA was extracted with RNeasy Micro Kit (Qiagen), according to manufacturer's instructions. RNA quality was assessed by an Agilent 2100 Bioanalyser (G2943CA), using an Agilent RNA 6000 Nano Kit (5067-1511). Gene expression was evaluated by a whole human genome microarray Human Gene 2.1 ST Array Strip from Affymetrix. The microarrays were scanned by a GeneAtlas system from Affymetrix. The raw data were analyzed using Expression Console™ Software from Affymetrix which uses RMA (Robust Multiarray Averaging). Differentially expressed genes were identified also using Affymetrix® Expression Console™ Software. It was considered as differentially expressed gene a variation equal or higher than 2-fold between the different conditions. Genes with adjusted values of P<0.05 were considered to be significant. Biological processes and signaling pathway activity scores were generated by mapping all expressed genes using a classification system, the PANTHER (protein annotation through evolutionary relationship) (http://www.pantherdb.org/). Biological processes with at least 2 differentially expressed genes and pathways with at least 5 differentially expressed genes were considered for analysis.

Mice. Male^{LmnaG609G/G609G}, and wild-type were used (Osorio F G, Navarro C L, Cadinanos J, Lopez-Mejia I C, Quiros P M, Bartoli C, Rivera J, Tazi J, Guzman G, Varela I, Depetris D, de Carlos F, Cobo J, Andres V, De Sandre-Giovannoli A, Freije J M, Levy N, Lopez-Otin C. Splicing-directed therapy in a new mouse model of human accelerated aging. Science translational medicine. 2011;3:106ra107). Animal studies were approved by the local <<comité d'éthique pour l'expérimentation animale>> (Marseille Animal Care Commitee, Protocol n° 96-21122012) and conformed to the Directive 2010/63/EU of the European parliament regarding the protection of animals used for experimental and other scientific purposes. Mouse vSMCs (mSMC) were prepared from thoracic aortas of 6 or 18 week-old mice. Briefly, after fat tissue removal around aortic region, aorta was dissected from its origin to the proximity of the diaphragm. Aortas from two mice were put into HBSS 1×, on ice then rinse once in HBSS. Aorta were digested 10 minutes at 37° C. in enzyme solution freshly prepared the day of isolation (Collagenase 1 mg/ml, Soybean Trypsin inhibitor 1 mg/ml, elastase 0.744 units/ml—Worthington biochemical—, penicillin/streptomycin 1%, HBSS 1×). Aortas were then washed off with warmed and equilibrated DMEM/F12 (20% FBS inactive, 100 IU/mL penicillin, 100 μg/mL streptomycin). Adventitia was strip off under the binocular microscope and aortas were opened longitudinally with scissors. Endothelial cell layer was removed by gently scrapping the inside of the vessel with a forceps. Aorta were placed into a new dish of enzyme solution and incubated at 37° C. for about one or two hours with regular check under microscope regarding cell dissociation. Cells were triturated with a fire polished Pasteur pipette and collected at 1.5 rpm during 5 min, washed twice in DMEM/F12 media and placed in 3 wells of a 48 well dish. After one week, media was replaced. Cells were grown in DMEM/F12 medium that contained 100 IU/mL penicillin, 100 μg/mL streptomycin, and 20% fetal bovine serum inactive at 37° C. in a humidified atmosphere at 5% CO2. mSMCs were used at passages 4 to 5. Cells were characterized for SMC and Progeria markers and cultured under flow conditions (120 dynes/cm2). The loss of mSMC during time was assessed by the percentage of occupied area.

In vivo studies. 16 Lmna^{G609G/G609G} mice (male and female) were used. After sex and bodyweight randomization, animals were allocated in different groups and treated with vehicle (8 Lmna^{G 609G/G 609G} control mice) or BB-94 inhibitor (8 Lmna^{G609G/G609G} mice treated with Batimastat in vehicle solution). IP injections were used to administrate 30 mg/Kg/day of BB-94 in 3 mg/ml in PBS containing 0.01% Tween 80. The treatment was applied 5 times per week during 6 weeks (from week 5 to week 10). The treatment duration was reduced from 10 to 6 weeks due to intra-abdominal massive accumulation of BB-94 (precipitate). At the end of week 10 the mice were sacrificed and the selected parameters were evaluated.

Gene Expression Profiling using Fluidigm. Small parts of mice aortas were frozen to be analyzed. These parts were lysated and homogenized using a MagNA instrument and MagNA Lyser Green Beads (with 5 mm (diameter) stainless steel beads). Total RNA were then isolated and quantified. DELTAgene assays (FlexSix—Fluidigm) were designed for human transcripts. The pre-amplification process was performed for 14 cycles in order to obtain sensitivity down to a single cDNA molecule. The oligos were synthesized by Sigma and dissolved at a concentration of 100 μM in water. For each assay a Primer Pair Mix was prepared containing 50 μM Forward Primer and 50 μM Reverse Primer. In order to prepare 10× Pre-amplification Primer Mix (500 nM each primer), 10 μL of each of the 96 Primer Pair Mixes (50 μM each primer) was mixed with 40 μL buffer consisting of 10 mM Tris-HCl, pH 8.0; 0.1 mM EDTA; 0.25% Tween-20. In order to prepare 10× Assay (5 μM each primer) each Primer Pair Mix was diluted by mixing 10 μL Primer Pair Mix (50 μM each primer) with 90 μL buffer consisting of 10 mM Tris-HCl, pH 8.0; 0.1 mM EDTA; 0.25% Tween-20. A pre-mix containing cDNA and primers was done and treatment with exonuclease I was performed to remove non-hybridized primers. The Fluidigm® FLEXsix™ Gene expression IFC was used with EvaGreen chemistry. After a prime of the chip, a 10× assay mix and sample mix were prepared and pipetted into the inlets. The chip was loaded and data was collected using the BioMark HD™. Data was analyzed using Fluidigm® Real Time PCR Analysis v2.1 software.

Statistical analysis—In an embodiment statistical analyses were performed with GraphPad Prism software. For multiple comparisons, a one-way ANOVA analysis with Newman-Keuls post-test was performed. Results were considered significant when P<0.05. Data are shown as mean±SEM unless other specification.

In an embodiment, it has been shown that wild-type mouse Lmna gene with a mutant allele that carried the c.1827C>T; p.Gly609Gly mutation recapitulate most of the described alterations associated with HGPS, including the loss of SMC (Osorio F G, Navarro C L, Cadinanos J, Lopez-Mejia I C, Quiros P M, Bartoli C, Rivera J, Tazi J, Guzman G, Varela I, Depetris D, de Carlos F, Cobo J, Andres V, De Sandre-Giovannoli A, Freije J M, Levy N, Lopez-Otin C. Splicing-directed therapy in a new mouse model of human accelerated aging. Science translational medicine. 2011;3:106ra107). Therefore to validate the results obtained for HGPS-iPSC-SMCs we isolated SMCs from wild-type mice (WT mSMC) and homozygous Lmna G609G/G609G (HOZ mSMC). Both cells, expressing calponin and α-SMA, were isolated from mice thoracic aortas at 6 weeks (FIG. 17A). HOZ mSMCs also show dysmorphic nuclei and nuclear blebbing (FIG. 17A and FIG. 17B). WT mSMC were cultured under flow conditions (120 dyne/cm2; to mimic mice arterial flow shear stress) for up to 26 days without visible loss of cells (FIG. 17C). On the other hand, HOZ mSMC detached from the substrate after 8-9 days. These results confirm that mSMC are vulnerable to flow shear stress and are in agreement with HGPS-iPSC SMC results.

Nuclear abnormalities such as dysmorphic nuclei and nuclei blebbing also peaked at day 4. The inhibition of progerin by antisense morpholinos (Osorio F G, Navarro C L, Cadinanos J, Lopez-Mejia I C, Quiros P M, Bartoli C, Rivera J, Tazi J, Guzman G, Varela I, Depetris D, de Carlos F, Cobo J, Andres V, De Sandre-Giovannoli A, Freije J M, Levy N, Lopez-Otin C. Splicing-directed therapy in a new mouse model of human accelerated aging. Science translational medicine. 2011;3:106ra107) decreased significantly the detachment of HGPS-iPSC SMCs (FIG. 18).

in an embodiment to quantify the level of MMPs in the culture media of HGPS-iPSC SMCs and N-iPSC SMCs after flow shear stress. The level of MMPs increased in the culture media of HGPS-iPSC SMCs but not in N-iPSC-SMC media (FIG. 19B.1). Remarkably, the level of MMP13 increased 30-fold in cell culture media of HGPS-iPSC SMCs but not in cell culture media of the other cell types tested (N-iPSC SMCs, hVSMCs or HGPS fibroblasts) (FIG. 19B.2). High MMP13 levels have been also obtained in media collected from HOZ mSMCs under flow shear stress but not in WT mSMCs (FIG. 19C).

Our results show that SMCs knockdown for MMP13 have increased stability in flow culture conditions than non-treated cells. According to our results the chemical inhibition of MMP13 in HGPS-iPSC SMCs cultured for 7 days in flow conditions reduce significantly the percentage of progerin positive cells (FIG. 20C). We also analyzed the effect of MMP13 and BB94 inhibition in HOZ mSMCs. Similar to what was observed with HGPS-iPSC SMCs, the detachment was significantly delayed when one of the inhibitors were used (FigureD). Overall, our results obtained from gain-loss function experiments indicate that MMP13 mediates SMC loss.

In an embodiment, inhibition of MMP13 in LmnaG609G/G609G mice significantly increased the number of SMCs in aortic arch. So far no therapy has been developed to target specifically SMC loss. Most of the compounds identified so far in pre-clinical tests to treat progeria have been focused in the reduction of progerin quantities, by either reducing its production or increasing its degradation, in the reduction of progerin toxicity by targeting its aberrant prenylation, or identifying compounds capable of restoring pathological phenotypes downstream of progerin accumulation. Therefore we asked whether the inhibition of MMP13 in LmnaG609G/G609G mice could retard SMC loss. For these studies we have used Batimastat since safety has been demonstrated in clinical trials. LmnaG609G/G609G mice (n=8 for treatment group and control group; age: 5 weeks) were IP injected 5 times a week (30 mg/Kg/day; 3 mg/mL in PBS). We had to terminate the study before the 10 weeks of treatment defined initially because of the low solubility of the compound and precipitation in the abdomen. At this time point we evaluated SMC loss in the aortic arch. Our results (cell nuclei counting and qRT-PCR results) clearly show that animals treated with Batimastat have higher number of SMCs than untreated animals (FIG. 21). No evidence of decrease of progerin and accumulation of progerin were found in the cells isolated from aortic arch.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

1. A method for treating Hutchinson-Gilford Progeria Syndrome or vascular ageing diseases comprising administering an effective amount of a metalloprotease inhibitor comprising a zinc endopeptidase, wherein said inhibitor is selected from the following compounds: pyrimidine-4,6-dicarboxylic acid, bis-(4-fluoro-3-methyl-benzylamide);
pyrimidine-4,6-dicarboxylic acid, bis-(3-methyl-benzylamide);
pyrimidine-4,6-dicarboxylic acid, bis-(benzylamide);
pyrimidine-4,6-dicarboxylic acid bis-[(Pyridin-3-YL-Methyl)-Amide;
(2R,3S)-N4-Hydroxy-2-isobutyl-N1-[(2S)-1-(methylamino)-1-oxo-3-phenyl-2-propanyl]-3-[(2-thienylsulfanyl)methyl]succinamide;
N-[2,2-dimethyl-1-(methylcarbamoyl)propyl]-2-[hydroxy-(hydroxycarbamoyl)methyl]-4-methyl-pentanamide;
N-hydroxy-4-((4-((4-hydroxy-2-butynyl)oxy)phenyl)sulfonyl)-2,2-dimethyl-3-thiomorpholinecarboxamide;
MMP-13 siRNA, and mixtures thereof,
to a patient in need thereof.

2. The method according to claim 1 wherein said inhibitor is an inhibitor of MMP-1, MMP-7, MMP-13 or MMP-14.

3. The method according to claim 1 wherein said inhibitor is an inhibitor of MMP-13.

4. (canceled)

5. The method according to claim 1 wherein said inhibitor is selected from the following compounds:
(2R,3S)-N4-Hydroxy-2-isobutyl-N1-[(2S)-1-(methylamino)-1-oxo-3-phenyl-2-propanyl]-3-[(2-thienylsulfanyl)methyl]succinamide;
N-[2,2-dimethyl-1-(methylcarbamoyl)propyl]-2-[hydroxy-(hydroxycarbamoyl)methyl]-4-methyl-pentanamide;
N-hydroxy-4-((4-((4-hydroxy-2-butynyl)oxy)phenyl)sulfonyl)-2,2-dimethyl-3-thiomorpholinecarboxamide;
MMP-13 siRNA; and mixtures thereof.

6. The method according to claim 1 wherein the pyrimidine-4,6-dicarboxylic acid, bis-(4-fluoro-3-methyl-benzylamide) is an inhibitor of MMP-13.

7. The method according to claim 5 wherein the (2R,3S)-N4-Hydroxy-2-isobutyl-N1-[(2S)-1-(methylamino)-1-oxo-3-phenyl-2-propanyl]-3-[(2-thienylsulfanyl)methyl]succinamide is an inhibitor of MMP-1, MMP-2, MMP-3, MMP-7 or MMP-9.

8. A method for treating smooth muscle diseases comprising administering an effective amount of a metalloprotease inhibitor wherein said inhibitor is selected from the following compounds:
pyrimidine-4,6-dicarboxylic acid, bis-(4-fluoro-3-methyl-benzylamide);
pyrimidine-4,6-dicarboxylic acid, bis-(3-methyl-benzylamide);
pyrimidine-4,6-dicarboxylic acid, bis-(benzylamide);
pyrimidine-4,6-dicarboxylic acid bis-[(Pyridin-3-YL-Methyl)-Amide;
(2R,3S)-N4-Hydroxy-2-isobutyl-N1-[(2S)-1-(methylamino)-1-oxo-3-phenyl-2-propanyl]-3-[(2-thienylsulfanyl)methyl]succinamide;
N-[2,2-dimethyl-1-(methylcarbamoyl)propyl]-2-[hydroxy-(hydroxycarbamoyl)methyl]-4-methyl-pentanamide;
N-hydroxy-4-((4-((4-hydroxy-2-butynyl)oxy)phenyl)sulfonyl)-2,2-dimethyl-3-thiomorpholinecarboxamide;
MMP-13 siRNA; and mixtures thereof.

9. A pharmaceutical composition comprising at least one metalloprotease inhibitor as described in claim 1 in a therapeutically effective amount and a pharmaceutically acceptable carrier, adjuvant, excipient, or mixtures thereof.

10. The composition according to claim 9, wherein the composition is an injectable formulation, in particular an intraperitoneal injection.

11. The composition according to claim 9, wherein the inhibitor concentration is between 5 nM-7000 nM.

12. The composition according to claim 9, wherein the inhibitor concentration is between 5 nM-240 nM.

13. The composition according to claim 9, wherein the inhibitor concentration is between 5 nM-100 nM.

14. The composition according to claim 9, wherein the inhibitor concentration is between5 nM-50 nM.

15. The composition according to claim 9, wherein the inhibitor concentration is between 8 nM-20 nM.

16. A kit for drug screening for the treatment or diagnosis of Hutchinson-Gilford Progeria Syndrome or for the treatment or diagnosis of vascular ageing diseases or for the treatment or diagnosis of smooth muscle cells diseases, comprising:
at least one metalloprotease inhibitor of claim 1;
a fluidic system suitable for screening therapeutic drugs; and
a Hutchinson-Gilford Progeria Syndrome smooth muscle cell population.
